(19) United States
(12) Patent Application Publication
(10) Pub. No.: US 2008/0154348 A1
(43) Pub. Date: Jun. 26, 2008

(54) MRI COMPATIBLE IMPLANTABLE DEVICES

(76) Inventors: Ergin Atalar, Columbia, MD (US); Ahmet Ermeydan, Ankara (TR)

Correspondence Address: ECKERT SEAMANS CHERIN & MELLOTT, 600 GRANT STREET, 44TH FLOOR, PITTSBURGH, PA, 15219, US

(21) Appl. No.: 11/959,045

(22) Filed: Dec. 18, 2007

Related U.S. Application Data
(60) Provisional application No. 60/870,563, filed on Dec. 18, 2006.

(51) Int. Cl. A61N 1/05

(52) U.S. Cl. 607/116

(57) ABSTRACT

The present invention is directed to a novel implantable lead design which ensures safe magnetic resonance imaging of patients with active metallic implants such as pacemakers, neurostimulators and implantable cardio defibrillators. It is known that radio frequency and gradient fields of the MRI scanners may induce harmful currents on the implant leads. The present invention provides for the use of semiconductor components such as transistors and diodes to prevent such undesired induced currents on the implant leads. Circuits on the implants are designed such that while the induction of currents is prevented, the desired signal transmission in between the implanted pulse generator and the body part is maintained.

### CROSS-REFERENCE TO RELATED APPLICATION

This application claims the benefit of U.S. Provisional Application No. 60/870,563, filed Dec. 18, 2006, entitled “MRI Compatible Implantable Devices,” which is incorporated herein by reference.

### BACKGROUND OF THE INVENTION

1. Field of the Invention

The present invention relates to implantable devices, such as, without limitation, pacemakers and, in particular, to mechanisms for resisting the induction of currents in the leads of such devices from an external electromagnetic field and therefore reduce the likelihood of excessive heating from such fields.

2. Description of the Prior Art

Magnetic resonance imaging (MRI) generally is regarded as an extremely safe, non-invasive diagnostic technique. MRI may, however, pose a threat to patients that have implantable devices, such as, without limitation, a deep brain stimulation (DBS) device, a pacemaker, a neurostimulator, or a cardio-defibrillator. Currently, patients with metallic implants are not allowed to undergo an MRI scan. One of the main reasons for this is the excessive heating caused by the electromagnetic field concentration around the leads of an implant during an MRI procedure.

Many cases with substantial temperature increase during MRI scanning have been reported and reviewed. For example, a maximum temperature increase of 63.1° C. has been reported during 90 seconds of MRI scanning (Achenbach, S. et al., Am. Heart J 1997, 134:467-473). Additionally, in an in vitro evaluation of 44 commercially available pacemaker leads, it has been reported that a temperature increase of 23.5° C. was observed in a 0.5 Tesla experiment (Sommer, T. et al., Radiology 2000; 215:869-879). Substantial temperature increases also have been observed in MRI scans involving patients with neurostimulators (Gleason, C. A. et al., Pacing Clin. Electrophysiolgy 1992, 15; 81-94). Furthermore, 1.5 T and a SAR of 3.0 W/kg have been shown to cause severe necrosis in the mucous membranes of dogs with transesophageal cardiac pacing leads (Hofman, M. B. et al., Magn. Reson. Med 1996, 35:413-422).

One study has a shown a 16.8° C. temperature increase on a half wavelength wire in a gel-phantom (Smith, C. D. et al., J. Applied Physics 2000, 87:6188-6190). Another study has shown temperature increases due to endovascular guide wires between 26° C. and 74° C. in saline bath experiments of up to 30 seconds of scan time (Konings, M. K. et al., J. Magn. Reson. Imaging 2000, 12:79-85); In another study using saline solution, up to 34° C. of temperature increase was observed for a half wavelength wire (Nitz, W. R. et al., J. Magn. Reson. Imaging 2001, 13:105-114). It should be noted that first, second or third order burns were observed in many of the in vivo studies mentioned above.

A recent study was performed for one of the most widely used neurostimulation systems, the Activa Tremor Control System sold by Medtronic, Inc. Different configurations were evaluated to assess worst case and clinically relevant positioning scenarios, and in vitro experiments were performed at 64 MHz MR system using gel phantoms to represent human tissue. As reported by Rezaim A. R. et al. (J. Magn. Reson. Imaging 2002, 15:241-250), the highest temperature change observed was 25.3° C. for the RF coil and 7.1° C. for the head coil. These results indicate that heating may be hazardous under certain MRI scanning conditions.

The FREEHAND System Implantable Functional Neurostimulator is a commercially available RF-powered motor control neuroprosthesis which consists of both implanted and external components sold by NeuroControl Corporation of Cleveland, Ohio. Findings from an MRI-induced heating experiment, during which the FREEHAND System was exposed to a whole-body-averaged SAR of 1.1 W/kg for 30 minutes, showed that localized temperature increases were no greater than 2.7° C. with the device in a gel-filled phantom. A patient with a FREEHAND system can thus only undergo an MRI procedure under certain input power levels for a 1.5 Tesla scanner.

Due to the safety concerns created by the potential for excessive heating as described above, several strategies have been developed to promote MRI safety for patients having metallic implants. One basic strategy is to set a power threshold which ensures that only a reasonable amount of heating will occur. A methodology for such a power limitation previously was published by Yeung, C. J. et al. (Magn. Reson. Med. 2002; 47:187-193). However, many modern MRI pulse sequences, such as fast spin-echo or steady-state free precession (SSFP), require high RF power levels and, therefore, there is no guarantee that good quality images can be acquired if RF power is limited.

RF chokes and filters have been used previously by several investigators. For example, Susil, R. C. et al, (MRM 47:594-600, 2002) have used RF chokes in the design of a combined electrophysiology/MRI catheter, and Ladd, M. E. et. al. (MRM 43:615-619, 2000) have used triaxial chokes to present high impedance to currents flowing on the outer surface of the triax.

U.S. Pat. No. 7,123,013 discloses an implantable medical device which incorporates a rectifier diode inserted into a conductive strand (i.e., lead). Such a diode is known as a “rectifier” since it passes only the positive portion of a sinusoidal RF current and blocks negative portions. The maximum current reduction that is obtained is about a factor of 2. This patent does not disclose, however, using an electronic switch or a resistive element in parallel with a diode. Because of this, a charge accumulation will occur in the capacitor that is typically used in all modern implants. To avoid this, the capacitor would need to be placed in series to the circuit, which the patent does not disclose. Therefore, a charge accumulation will eventually reach a level that would inhibit the implant function. Because this patent does not disclose using a series capacitor, a polarization at the lead can occur and cause several problems. One of the most serious problems is the quick discharge of the battery of the implant.

U.S. Pat. No. 6,539,253 discloses implantable medical devices which incorporate integrated circuit notch filters. U.S. Pat. No. 5,817,136, discloses a pacemaker with EMI protection. Both of the above-disclosed designs ensure that electromagnetic interference to the implant does not occur. However, these designs do not guarantee safety with regard to lead heating. This is because high currents may still flow through long cables and these high currents may cause excessive heating and burns.

U.S. Pat. No. 5,217,010 discloses optical signal transmission in between the generator and the body part, such as Biophan's “photonic pacemaker.” This type of pacemaker has been shown to be safe (Greatbatch, W. et al., J. Magn. Reson. Imaging 2002, 16:97-103), because there is no coupling with the optical system and the electromagnetic field. However, the electrical to optical and optical to electrical energy conversion efficiency is limited and, therefore, the lifetime of the pulse generator reduces significantly. Miniaturization of the device also is a difficult task.

Another possible safety problem with MRI is that gradient-induced currents on the implants may cause undesired nerve stimulation with the possibility of cardiac arrest. A spinal Fusion Stimulator was analyzed theoretically for this purpose by Reilly, J. P. et al. (Magn. Reson. Imaging, 1997; 15(10):1145-56) and, although the authors noted a reduction in the stimulation threshold, the change in its level was not alarming.

Based on the foregoing, there exists a need for an implantable device which resists the induction of currents from an external electromagnetic field, such as the field that is present during MRI scanning, which, therefore, reduces the likelihood of excessive heating from such fields.

### SUMMARY OF THE INVENTION

The present invention meets this need by providing an apparatus that may be implanted within a patient's body that resists the induction of a current in one or more leads of the apparatus from an electromagnetic field external to the apparatus.

The apparatus includes electronic circuitry in which one or more leads are operatively coupled to the electronic circuitry. The one or more leads includes one or more electrical wires. The one or more leads also includes one or more active blocking circuits, which are responsible for resisting the induction of a current from an electromagnetic field. The active blocking circuits are comprised of one or more electronic switches. Examples of active blocking circuits include, without limitation, certain diodes that may function as an electronic switch (rather than merely as a rectifier), such as PIN diodes, or transistors. The electronic circuitry is comprised of, without limitation, an implantable pulse generator (IPG) for generating one or more electrical pulses, in which each of the one or more leads operatively coupled to the electronic circuitry delivers one or more of the electrical pulses to tissue within a patient's body.

In an embodiment of the present invention, the apparatus comprises an IPG which includes one or more active blocking circuits comprised of a PIN diode as the electronic switch in parallel with a resistor. The IPG is operatively coupled to one lead, which is operatively coupled to an electrode. The lead is comprised of four wires in which each of the four wires includes one of the one or more active blocking circuits.

In another embodiment of the present invention, the apparatus comprises an IPG that is operatively coupled to four leads, in which each of the four leads is operatively coupled to an electrode. Each of the four leads is comprised of two wires in which each of the two wires includes one of the one or more active blocking circuits.

In a further embodiment of the present invention, the apparatus comprises an IPG which includes one or more active blocking circuits comprised of a resistor in parallel with a series combination of a first one of the one or more electronic switches and a second one of the one or more electronic switches, in which the first electronic switch is a diode and the second electronic switch is a transistor. The IPG is operatively coupled to one lead, in which the lead is operatively coupled to an electrode. The lead is comprised of four wires, in which each of the four wires includes one of the one or more active blocking circuits. Each of the four wires is operatively coupled to the transistor of the active blocking circuit of another one of the four wires.

In another embodiment of the present invention, the apparatus comprises an IPG which is operatively coupled to four leads in which each of the four leads is operatively coupled to an electrode. Each of the four leads is comprised of two wires, in which each of the two wires includes one of the one or more active blocking circuits. Each of the two wires is operatively coupled to the transistor of the active blocking circuit of another one of the two wires from a different one of the four leads.

In a further embodiment of the present invention, the apparatus comprises an IPG which is operatively coupled to one lead which is operatively coupled to an electrode. The one lead is comprised of four signal wires and four control line wires, in which each of the four signal wires includes one of the one or more active blocking circuits. Each of the control line wires is operatively coupled to the transistor of a respective one of the one or more active blocking circuits.

In another embodiment of the present invention, the apparatus comprises an IPG which is operatively coupled to four leads, in which each of the four leads is operatively coupled to an electrode. Each of the four leads is comprised of two signal wires and two control line wires, in which each of the two signal wires includes one of the one or more active blocking circuits. Each of the control line wires is operatively coupled to the transistor of a respective one of the one or more active blocking circuits.

In a further embodiment of the present invention, the apparatus comprises an IPG which includes at least one of the one or more leads being operatively coupled to an electrode, in which the one of the one or more leads includes a first wire, a second wire and a control line wire. The first wire includes a resistor in series with a transistor and the second wire includes a resistor. A capacitor is operatively coupled between the first wire and the second wire. The control line wire is provided between the electronic circuitry of the apparatus and the transistor.

In another embodiment of the present invention, the apparatus comprises an IPG which includes at least one of the one or more leads being operatively coupled to an electrode, in which the one of the one or more leads includes a first wire and a second wire. The first wire includes a resistor and a capacitor in series and the second wire includes a resistor. A transistor is operatively coupled between the first wire and the second wire.

In a further embodiment of the present invention, the apparatus includes electronic circuitry for operating the apparatus, in which a case surrounds the electronic circuitry. One or more leads are operatively coupled to the electronic circuitry. An electronic switch is provided between the electronic circuitry and the case for resisting the induction of a current from the electromagnetic field. The electronic circuitry may include, without limitation, an IPG for generating one or more electrical pulses, in which each of the one or more leads delivers one or more electrical pulses to tissue within a patient's body.

In another embodiment of the present invention, there is provided a method of operating an implantable device which resists the induction of a current from an electromagnetic field external to the device. The device includes electronic circuitry for operating the device and one or more leads operatively coupled to the electronic circuitry. Each of the one or more leads is operatively coupled to a respective electrode. The method comprises periodically providing an electrical signal to a patient through each of the one or more leads and the electrode operatively coupled thereto; and electrically isolating each of the electrodes from the electronic circuitry during a period in which the electrical signal is not being provided to the patient through each of the one or more leads. Each of the electrodes may be electrically isolated from the electronic circuitry by employing an active blocking circuit.

### BRIEF DESCRIPTION OF THE DRAWINGS

The accompanying drawings illustrate presently preferred embodiments of the invention, and together with the general description given above and the detailed description given below, serve to explain the principles of the invention. As shown throughout the drawings, like reference numerals designate like or corresponding parts.

FIG. 1 is a schematic diagram of a unipolar pacing model of a stimulator;

FIG. 2 is a schematic diagram of a bipolar pacing model of a stimulator;

FIG. 3 is a schematic diagram showing the mechanism of tip heating, in which the lead tip impedance, Rₜ, typically is approximately 1 kilo-ohm;

FIG. 4 is a schematic diagram of an insulation model of an IPG with high impedance inductors at 64 MHz, which is the operating frequency of 1.5T MRI scanners according to an embodiment of the present invention;

FIG. 5 is a schematic diagram of a diode resistor circuit (DRC) according to an embodiment of the present invention;

FIG. 6 is a schematic diagram of a two directional pacing DRC according to an embodiment of the present invention;

FIG. 7 is a schematic diagram of a multi-electrode two directional pacing DRC according to an embodiment of the present invention;

FIG. 8 is a schematic diagram of a transistor diode circuit (TDC) according to an embodiment of the present invention;

FIG. 9 is a schematic diagram of one type of a two directional pacing TDC according to an embodiment of the present invention;

FIG. 10 is a schematic diagram of one type of a multi-electrode two directional pacing TDC according to an embodiment of the present invention;

FIG. 11 is a schematic diagram of a second type of a two directional pacing TDC according to an embodiment of the present invention;

FIG. 12 is a schematic diagram of a second type of a multi-electrode two directional pacing TDC according to an embodiment of the present invention;

FIGS. 13A and 13B are schematic diagrams of a Capacitor Switch Capacitor (CSC) implementation model, in which FIG. 13A includes a parallel capacitor and FIG. 13B includes a series capacitor according to embodiments of the present invention;

FIG. 14 is an efficiency plot of CSC as a function of R₁ according to an embodiment of the present invention;

FIG. 15 is an efficiency plot of CSC as a function of capacitance according to an embodiment of the present invention;

FIG. 16 is an efficiency plot of CSC as a function of R₁ with an interval of 45 kilo-ohms to 55 kilo-ohms according to an embodiment of the present invention;

FIG. 17 is an efficiency plot of CSC as a function of t₁ according to an embodiment of the present invention;

FIG. 18 is an efficiency plot of CSC as a function of t₂ for C=10 μF, R₁=50 kilo-ohms, t₁=1 sec and R₂=1 kilo-ohm according to an embodiment of the present invention;

FIG. 19 shows a simulation result of a DRC on target body resistance according to an embodiment of the present invention;

FIGS. 20A and 20 B show two simulation results of TDC for unipolar pacing according to an embodiment of the present invention;

FIG. 21 shows a simulation result of a TDC for bipolar pacing according to an embodiment of the present invention;

FIG. 22 shows a simulation result of CSC for bipolar pacing according to an embodiment of the present invention;

FIGS. 23A and 23B show simulation results of CSC (parallel capacitor) on the tip (23A) and on the ring (23B) according to an embodiment of the present invention;

FIG. 24 shows a simulation result of CSC (series capacitor) on the tip and the ring according to an embodiment of the present invention;

FIG. 25 shows a simulation result of TDC on the tip according to an embodiment of the present invention;

FIGS. 26A and 26B are illustrations of experimental setups without (26A) and with (26B) CSC;

FIG. 27 is an illustration of the sciatic nerve bundle of a frog's leg and shows where the sciatic nerve exits the vertebral column;

FIG. 28 is an illustration of a frog's leg sciatic nerve in thigh musculature;

FIG. 29 is a graphical plot showing the result of a heating experiment of an IPG model without CSC according to an embodiment of the present invention;

FIG. 30 is a graphical plot showing the result of a heating experiment of an IPG model with CSC according to an embodiment of the present invention; and

FIG. 31 is a graphical plot showing the result of a heating experiment of an IPG model with DRC according to an embodiment of the present invention.

### DESCRIPTION OF THE PREFERRED EMBODIMENTS

The present invention provides an MRI compatible implantable apparatus that can be used safely with magnetic resonance imaging (MRI). The implantable apparatus of the present invention is comprised of one or more leads that includes one or more active blocking circuits, in which each of the one or more active blocking circuits includes one or more electronic switches (e.g., without limitation, certain types of diodes or transistors). The implantable apparatus effectively decreases the temperature rise that typically occurs at the tip (electrode) of the one or more implant leads. The apparatus of the present invention not only is easier to manufacture and to miniaturize, but also is more effective in reducing induced currents than prior art passive circuits such as choke inductors.

In implantable devices, such as implantable pulse generators (IPGs), there are two main pacing types: unipolar and bipolar. The unipolar pacing method uses only one wire in the pacing lead. The pacing pulse is transmitted by applying a potential between the pacing lead and the case that surrounds the IPG.

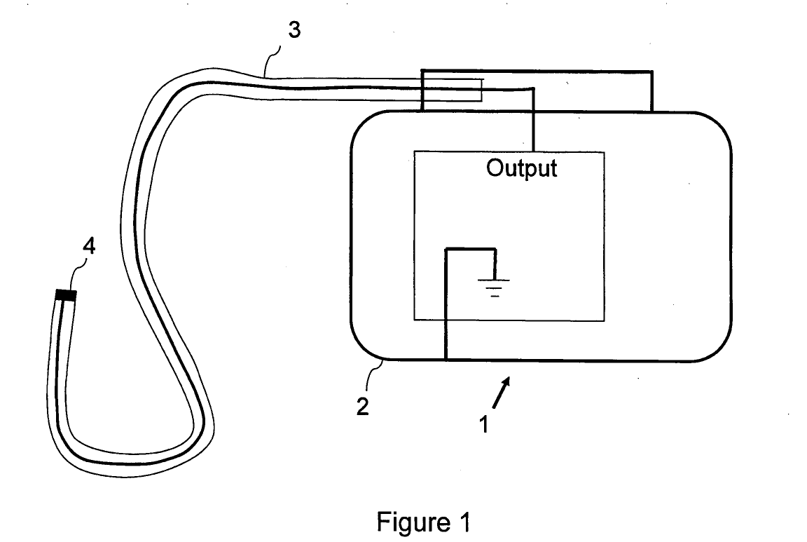

FIG. 1 is a schematic diagram of a unipolar pacing model of a stimulator, which shows a pacing lead 3, a tip 4 of the pacing lead 3, and an IPG 1 surrounded by a case 2.

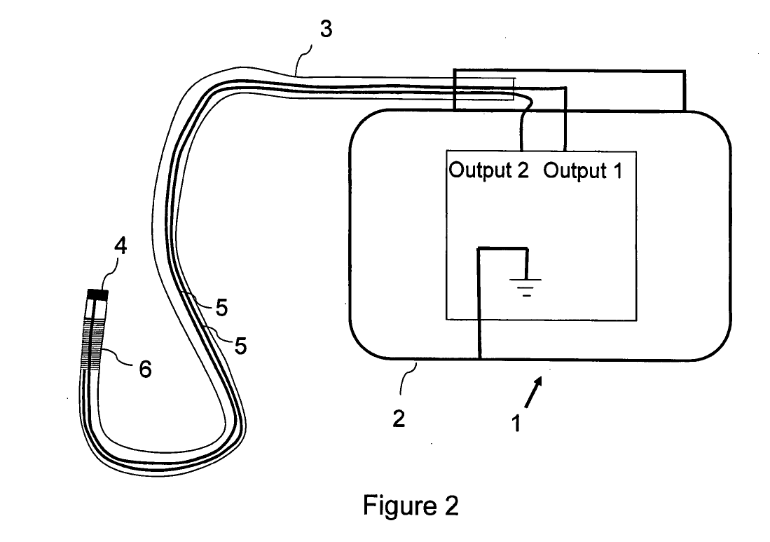

FIG. 2 is a schematic diagram of a bipolar pacing model of a stimulator, which shows the use of two wires 5 in the pacing lead 3, in which one wire 5 terminates in an electrode tip 4 and is used as the pacing signal and the other wire 5 includes a ring 6 and is used as a ground.

In accordance with a patient's need, both pacing techniques may be used at different time periods. Thus, advanced stimulators typically use both unipolar and bipolar pacing techniques. In addition, some stimulators, such as deep brain stimulators, use more than two wires. In these multi-wire arrangements, each of the wires can be programmed to act as a ground, a unipolar electrode or a bipolar electrode.

The heating mechanism that typically occurs during MRI can be explained as follows. In MRI, radio frequency (RF) pulses are applied to obtain echo from the sample of interest. The frequency of an RF pulse is proportional with the strength of the main magnetic field, which is approximately 64 MHz for a 1.5T MRI scanner. Although only a magnetic field is required to obtain the desired echo, because it is not possible to uncouple electric and magnetic fields, an electric field is generated in a patient's body. Optimization of field distribution that minimizes the electric field while keeping the magnetic field uniform in the body is achieved differently for different types of magnets. For example, with a horizontal bore magnet, the optimum field distribution is achieved by a special coil type, referred to as a birdcage coil. In a properly designed birdcage coil, the electric field at the center of the object is zero and increases linearly in the radial direction, while the electric field is oriented in the z-direction (along the axis of the magnet bore).

When a metallic implant is placed inside a patient's body, the electromagnetic field that is generated may induce a current on wires that are contained within the lead of the implant. The highest amount of current is generated when the lead is directed mostly in the z-direction and loops are formed by the lead.

The apparatus of the present invention broadly is comprised of two parts: an implantable device, such as an IPG, and one or more leads. The IPG typically is surrounded by a metallic case. The one or more leads may be insulated with a plastic coating. Each of the one or more leads terminates in a bare tip which touches the target organ. In some designs, such as bipolar leads, two wires are used in each lead, in which one of the wires is connected as a ground and connects to a relatively large metallic ring. In other designs, more than two wires are used and connected to a series of electrodes. In still other designs, multiple leads are used.

For example, when one lead with one electrode is used in a pacemaker, the electrical and magnetic field that is induced in a patient's body generates a potential difference (voltage) between the case of the pacemaker and the electrode tip. This voltage causes a current flow on the lead. This current typically is not high enough to cause heating of the wires of the lead, but the current leaves the lead at the bare tip of the electrode. A high concentration of such a current significantly heats up tissue adjacent to the tip.

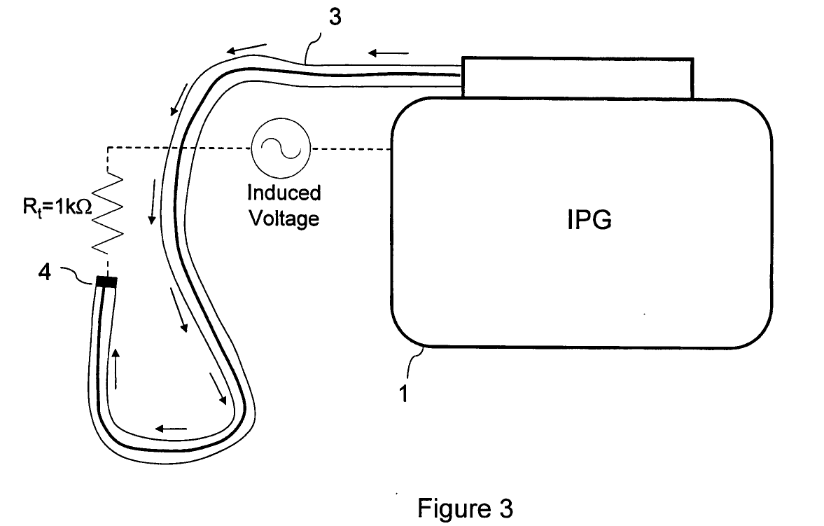

FIG. 3 is a schematic diagram of the mechanism of heating in an electrode tip 4 of a lead 3 that occurs in an IPG 1. Calculation of the amount of heating at the tissue adjacent to the lead tip 4 is a complex procedure. It involves solving an electromagnetic scattering problem in a lossy medium and also a bioheat problem in an inhomogeneous medium. Roughly, induced voltage on the wire results in a one kilo-ohm impedance at the tip.

In this type of circuit, other than lead resistance, there are other impedances in the current loop, such as lead impedance, lead-to-IPG case impedance and IPG to body impedance. In a typical implant design, induced current is determined by lead impedance and the other impedances are insignificant.

In order to block induced current, several methods have been previously developed. However, there are two important restrictions on such methods. First, the implant needs to continue to operate. Second, the mechanical properties of the implant should not be affected. The implantable apparatus of the present invention effectively blocks the current generated by MRI while maintaining the desired mechanical and electrical properties of the implant.

As previously described, the amount of induced current determined by the lead tip impedance, Rt, is approximately 1 kilo-ohm. This current can be reduced significantly by insulating the case of an IPG with a non-conducting medium, such as plastic. Although this approach is suitable for bipolar pacing designs, insulation of the case of the IPG eliminates the possibility of using the case as a ground in unipolar IPG designs.

In a unipolar design, the ground of the electronic circuitry of an apparatus, such as an IPG, is connected to the case of the apparatus through a high impedance inductor. The value of inductance needs to be selected such that at the operating frequency of the MRI system (1.5T scanners operating at 64 MHz), the impedance of the inductance is significantly higher than Rt. A typical acceptable value will be higher than 10 kilo-ohms. This corresponds to 250 micro-Henry for a 1.5T scanner. Higher impedances are desired but may be limited by the size of the inductor that can be placed in an IPG.

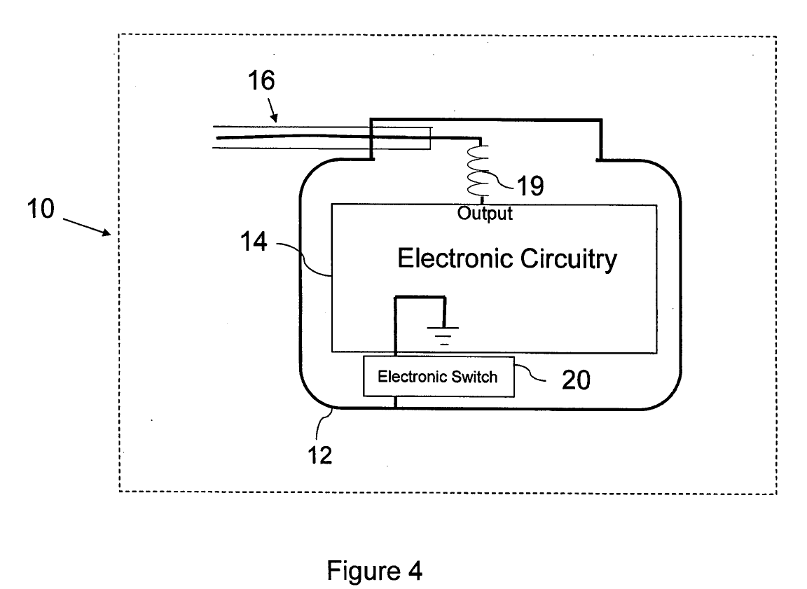

FIG. 4 is a schematic diagram of an insulation model of an implantable device according to one embodiment of the present invention. In particular, FIG. 4 shows an implantable apparatus 10 that includes electronic circuitry 14 and a case 12 surrounding the implantable apparatus 10. The electronic circuitry 14 and the case 12 are connected to one another through an electronic switch 20, such as, without limitation, a transistor. The ground connection to the case 12 of the implantable apparatus 10 is needed only during pacing, which is a very limited time (approximately 1 msec every 1 sec). Therefore, if the connection is established during only desired periods, heating of the tip 30 of a lead 16 of the implantable apparatus 10 can be reduced significantly. Control of the timing of the electronic switch 20 can be achieved by using particular circuitry in the implantable device 10. The design of such circuitry is well known by those skilled in the art. In addition, a high impedance inductor (not shown) may replace the electronic switch 20 or be placed in series with the electronic switch 20.

The induced currents can be reduced further by placing a simple circuit comprised of a high impedance series inductor 19 between the electronic circuitry 14 and a lead 16 of the implantable apparatus. Similar to the ground high impedance inductor that may be included between the electronic circuitry 14 and the case 12, this high impedance series inductor 19 has a significantly higher impedance than Rt in order to reduce the induced current. The value of the high impedance series inductor 19 may be adjusted so that it will not affect normal operation of the implantable apparatus 10. When multiple leads or leads with multiple conductors are used, blocking inductors may be used in each of them.

If an electronic switch 20 is used in place of a high impedance inductor 19 of the implantable apparatus 10, the electronic switch 20 connects the lead 16 to the electronic circuitry of the implantable apparatus 10 only when necessary and disconnects all other times. Most electronic switches have some current leakage which may be high at the MRI operating frequency if an improper electronic switch is selected. In the design of the electronic switch circuit of the present invention, radio frequency characteristics need to be analyzed and the value of the switch turn-off impedance should be adjusted such that it is significantly higher than the lead tip impedance of Rt. Achieving high impedance between the electronic circuitry 14 of the implantable apparatus 10 and the case 12 of implantable apparatus 10 reduces the heating of the tip significantly. However, radio frequency currents still may flow from the lead 16 directly to the patient's body by a mechanism known as displacement current. In this mechanism, the lead insulation material acts as a dielectric and high currents still may flow. This effect is well known by those skilled in the art.

The most effective current blocking typically is achieved at the tip of an implant lead. Using passive currents, shielded inductors at the tip have been proposed previously by the inventors and have been shown experimentally to be very effective. However, a critical issue is size limitation. In order to block the current, large inductors usually are necessary. Achieving the desired flexibility is possible but the manufacturing cost is increased significantly.

According to further embodiments of the present invention, the leads of an implantable device are provided with blocking circuits (described elsewhere herein) which include at least one electronic switch with the aim of reducing the problem of lead tip heating. As used herein, the term “electronic switch” shall include any circuit or circuit element that is able to perform a switching action such as, without limitation, a transistor or certain types of diodes, such as PIN diodes, that are capable of functioning as a switch. The term “electronic switch” shall not include a simple rectifier diode. This new approach is much more simple than the use of passive circuits. The electronic circuitry described herein can be miniaturized, and thus can be incorporated with flexible leads without significant effort and cost.

The present invention provides three different main embodiments, referred to as “Diode-Resistor Circuit (DRC)”, “Transistor-Diode Circuit (TDC)” and “Capacitor-Switch Circuit (CSC)”.

The first main embodiment, DRC, is comprised of a parallel electronic switch type diode, preferably a PIN-diode, and resistor placed in series to pacing leads to reduce induced current on the leads. Pacing energy is slightly reduced due to the finite turn-on voltage of the diode. However, induced RF current is reduced significantly because the PIN diode acts as a high valued resistor. The resistor in the circuit increases the leakage currents which alleviates the problem of charge accumulation at the tip. This design can be used in both unipolar and bipolar pacing.

In the second main embodiment, TDC, a resistor is placed on pacing leads in parallel to a series combination of a transistor and a diode (a simple rectifier diode) in order to reduce induced current on the leads from, for example, an MRI. Similar to the first embodiment, this circuit enables a pacing pulse to be transferred without significant attenuation while induced currents are blocked. This design can be used both in unipolar and bipolar pacing techniques.

In the third main embodiment, CSC, resistors are added on to pacing leads to reduce induced current on the leads. Pacing energy is accumulated in a series or a parallel capacitor at the distal end of the lead and discharged to the target body part with the aid of an electronic switch. Because the capacitor is charged with a very low current, impedance of the wire can be made very high.

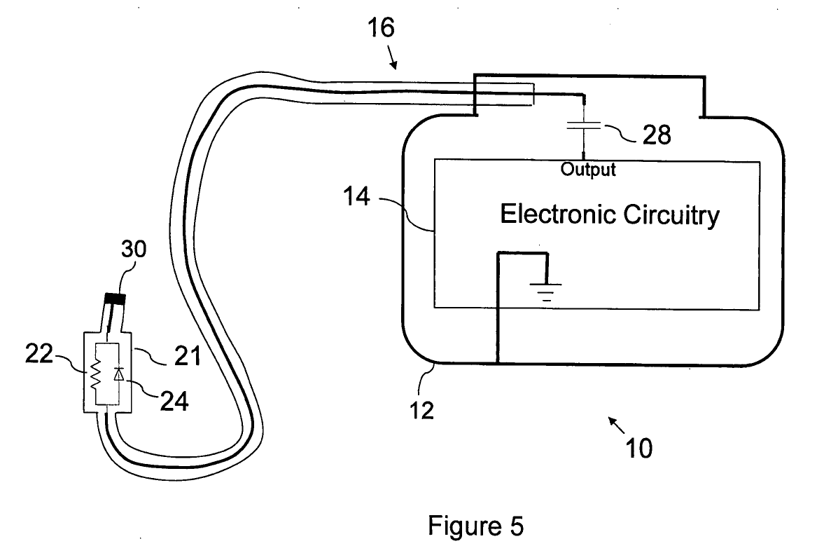

FIG. 5 is a schematic diagram of the DRC embodiment of the present invention, which shows an IPG 10, which is surrounded by a case 12 and includes electronic circuitry 14. A pacing lead 16 that terminates in an electrode tip 30 includes a capacitor 28 and an active blocking circuit 21. The active blocking circuit 21 includes a parallel combination of a PIN diode 24 and a resistor 22 to reduce induced current on the lead 16. During a pacing pulse, the PIN diode 24 conducts current after a pacing pulse level passes turn-on voltage threshold level (typically 0.6V) of the PIN diode 24. Therefore, there is no significant change in the applied signal level. When the stimulator is exposed to RF electromagnetic radiation, induced RF voltage on the lead 16 will not cause excessive current at the tip 30 of the lead 16 because of the PIN diode 24 and the resistor 22 pair. Although PIN diodes behave like regular diodes when a low frequency signal, such as a pacing signal, is applied, they behave like a resistor at radio frequency. Typically, their RF impedance is high (approximately 20 KΩ) when zero or negative bias is applied. Electrical properties of PIN diodes vary and can be obtained from their manufacturers.

One of the main characteristics of the DRC embodiment of the present invention is that, in normal operation, i.e., when an implantable apparatus sends a pacing pulse, it causes only a small loss of power. On the other hand, it exhibits a resistance to RF signals. This resistance is a function of an applied positive current. When no voltage or negative voltage is applied to a PIN diode, the RF resistance is in the order of several kilo ohms. This resistance is a function of frequency but typically is constant after a certain cut-off frequency. This cut-off frequency can be decreased by applying a reverse voltage to a PIN diode. This is a very useful property in order to block induced current flow on a lead. In the DRC embodiment of the present invention, the parallel PIN diode-resistor pair totally blocks induced current flow.

Standard stimulators use serial capacitors, such as capacitors 28 shown in FIG. 5, on each lead for two important reasons. First, serial capacitors provide a safety condition to block DC current flow to the target body part in a fault condition. Second, after a pacing pulse, some charge accumulates on the body. The serial capacitors null the total charge on the body when it is discharged. In order to discharge a series capacitor, a parallel resistor is used in the DRC embodiment of the present invention. The value of this parallel resistor needs to be chosen carefully. If resistance is too high, the capacitor cannot be discharged during one pacing cycle and will adversely affect the pacing capability of the DRC embodiment. On the other hand, if the resistance is too low, some RF current may flow in the resistor and the safety performance of the lead will diminish.

As an alternative to the DRC embodiment shown in FIG. 5, an inductor in series with a resistor, or replacing the resistor with an inductor with a high series equivalent resistor, may be used. In order to eliminate additional current induction to the inductor, a shield may be necessary. The shield may cover only the inductor or the inductor and a diode. As a further alternative, no resistors are necessary if a high reverse leakage current diode is used. In typical commercial diodes, leakage currents are very low and may not be suitable for this purpose.

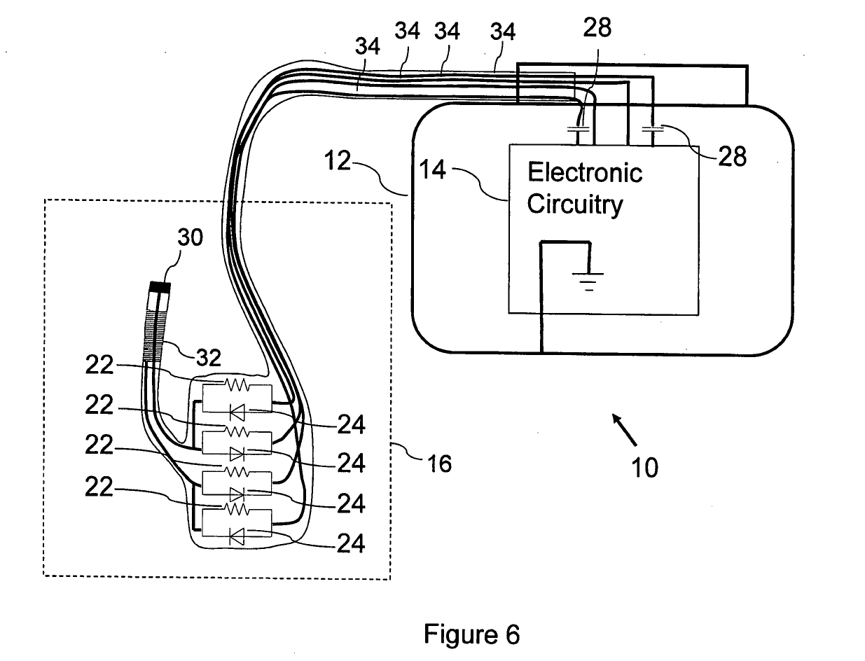

FIG. 6 is a schematic diagram of a particular two directional pacing DRC embodiment of the present invention, which shows an IPG 10 surrounded by a case 12 and includes electronic circuitry 14. In order to use both pacing directions for unipolar and bipolar pacing, the electronic circuitry 14 is operatively coupled to one lead 16 that terminates in an electrode tip 30. The lead 16 is comprised of four wires 34 (as seen in the Figures provided herein, certain of such wires 34 are electrode wires and certain of such wires 34 are ground wires), in which each of the four wires 34 includes an active blocking circuit 21 that includes a PIN diode 24 in parallel with a resistor 22.

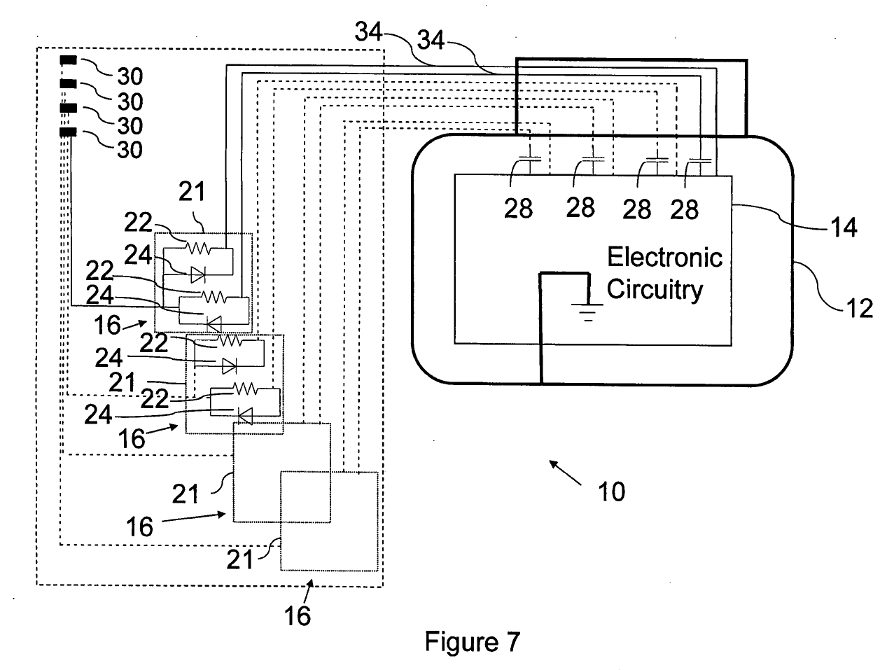

FIG. 7 is a schematic diagram of a particular multi-electrode two directional pacing DRC embodiment of the present invention, which shows an IPG 10 surrounded by a case 12. The IPG 10 is comprised of electronic circuitry 14 operatively coupled to four leads 16, each of the four leads terminating in an electrode tip 30. Each of the four leads 16 includes two wires 34, in which each of the wires 34 includes an active blocking circuit 21 that includes a PIN diode 24 in parallel with a resistor 22.

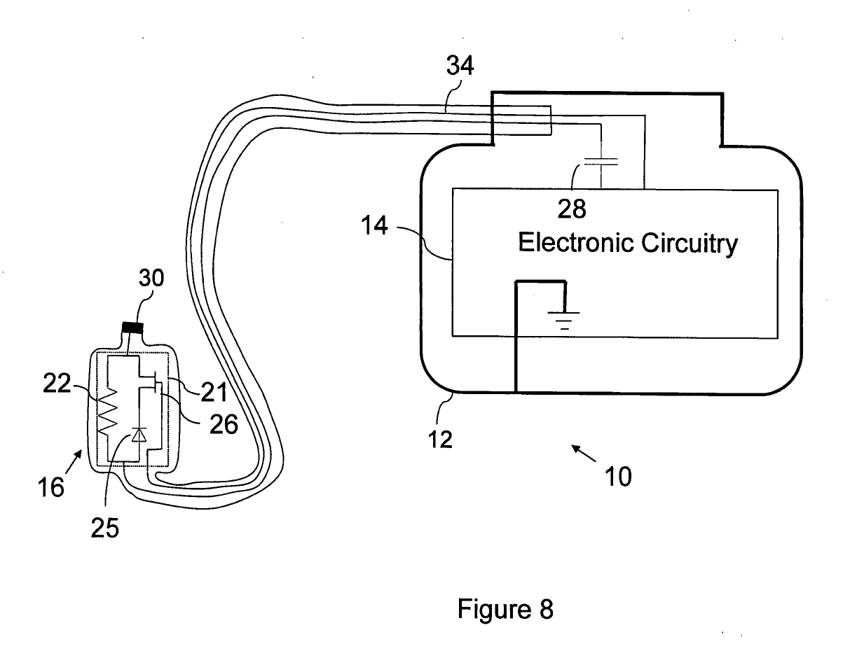

FIG. 8 is a schematic diagram of a particular TDC embodiment of the present invention, which shows an IPG 10 surrounded by a case 12, in which the IPG 10 is comprised of electronic circuitry 14 and one lead 16 operatively coupled to the electronic circuitry 14. The lead 16 includes two wires 34 and an active blocking circuit 21 comprised of a resistor 22 placed in parallel with a series combination of a transistor 26 and a diode 25 (preferably a normal, rectifier diode). The active blocking circuit 21 is included in the pacing lead 16 to reduce the induced current on the lead 16 in the presence of RF energy during an MRI procedure.

During a pacing pulse, the diode 25 conducts a current after the pulse level passes the threshold level of the diode 25. The transistor 26 controls current flow on the lead 16 by control signaling. By adjusting a suitable control signal, the transistor 26 conducts the pacing pulse without significantly changing the signal level. It is important that proper components are selected, for example, the diode 25 employed should be selected so as not to leak RF currents.

One of the important advantages of this particular embodiment over the DRC embodiment is that the TDC embodiment not only blocks induced RF currents but also blocks induced low frequency currents. Therefore, possible gradient-induced currents also will be blocked.

As with the DRC embodiment, a shunt resistor 22 is used in the TDC embodiment. A capacitor 28 in the IPG 10 discharges over the resistor 22. As discussed for the DRC embodiment, if the leakage current of the transistor 26 and the diode 25 pair is adjusted to a desired level, the resistor 22 may be eliminated.

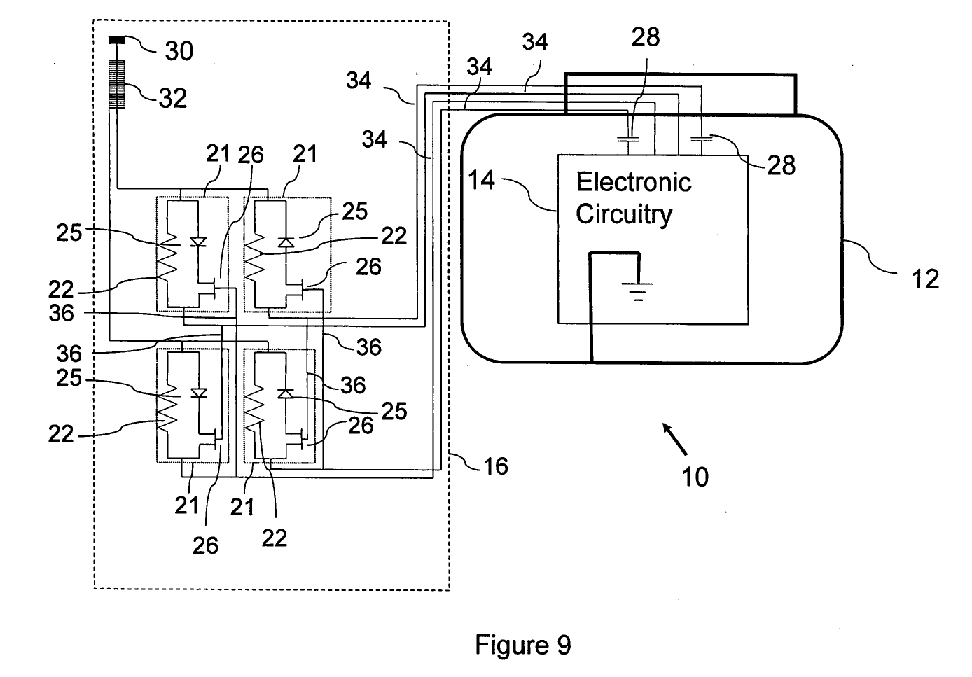

FIG. 9 is a schematic diagram of a particular two directional pacing TDC embodiment of the present invention which allows for the use of both unipolar and bipolar pacing. As shown in FIG. 9, there is provided an IPG 10 surrounded by a case 12, in which the IPG 10 is comprised of electronic circuitry 14 operatively coupled to one lead 16 which terminates in a ring 32 and an electrode tip 30. The lead 16 includes four wires 34, in which each of the four wires 34 includes an active blocking circuit 21 comprised of a resistor 22 in parallel with a series combination of a diode 25 and a transistor 26. Each of the four wires 34 is operatively coupled to the transistor 26 of the active blocking circuit 21 of another one of four wires 34 with control lines 36. The control lines 36 give an additional flexibility to the implantable apparatus 10.

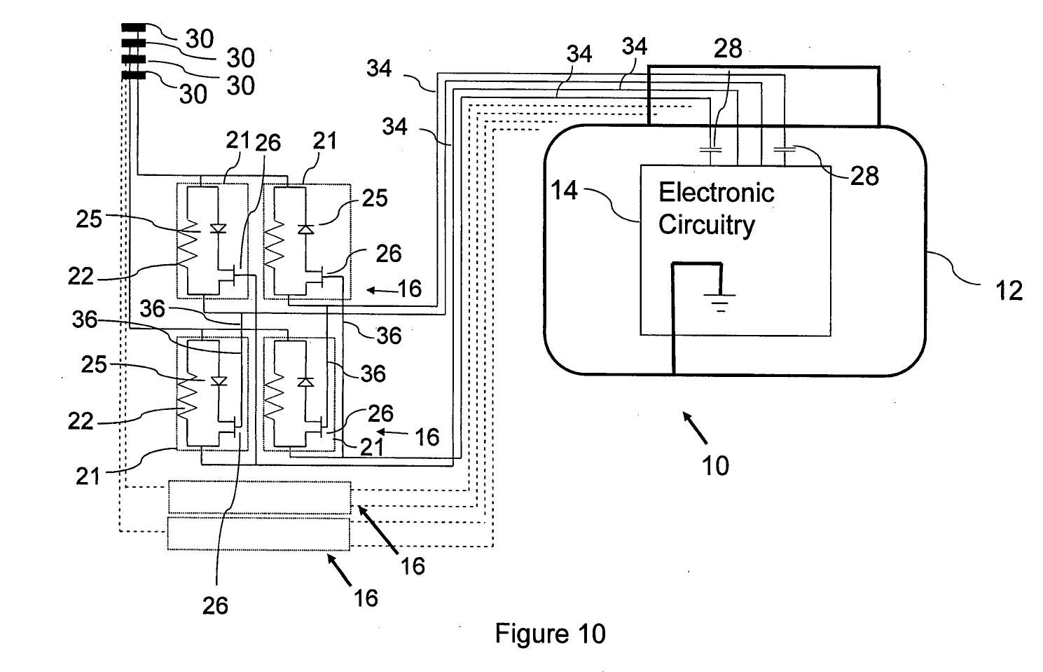

FIG. 10 is a schematic diagram of a particular multi-electrode two directional pacing TDC embodiment of the present invention, which shows an IPG 10 surrounded by a case 12, in which the IPG 10 is comprised of electronic circuitry 14 operatively coupled to four leads 16. Each of the four leads 16 terminates in an electrode tip 30. Each of the four leads 16 is comprised of two wires 34, in which each the two wires 34 includes an active blocking circuit 21 comprised of a resistor 22 in parallel with a series combination of a diode 25 and a transistor 26. Each of the two wires 34 is operatively coupled to the transistor 26 of the active blocking circuit 21 of another one of the two wires 34 from a different one of the four leads 16.

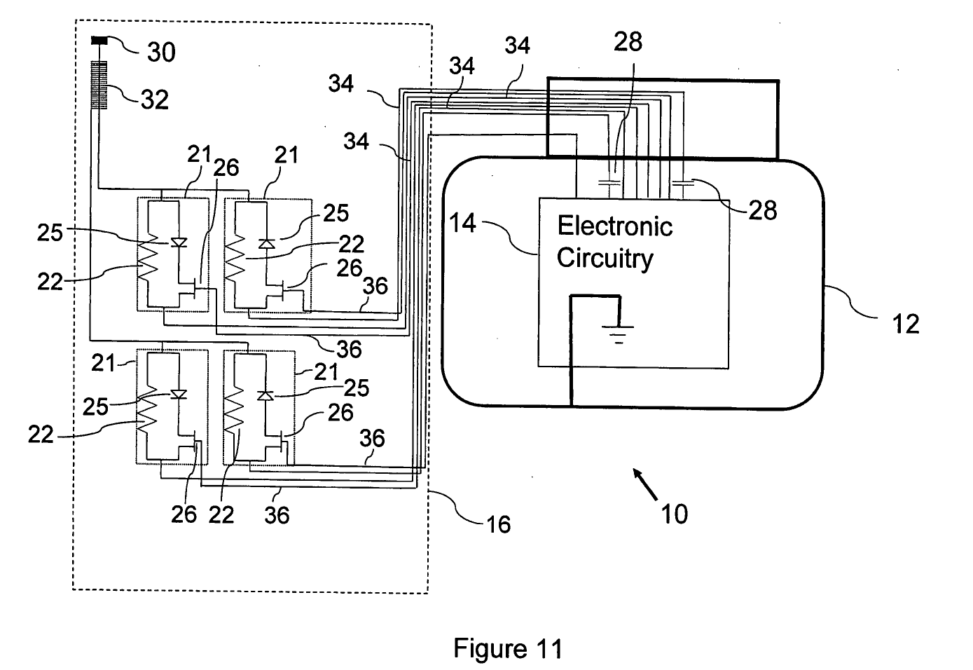

FIG. 11 is a schematic diagram of a particular two directional pacing TDC embodiment of the present invention, which shows an IPG 10 surrounded by a case 12, in which the IPG 10 is comprised of electronic circuitry 14 operatively coupled to one lead 16, which terminates in a ring 32 and an electrode tip 30. The lead 16 includes four signal wires 34 and four control line wires 36, in which each of the four signal wires 34 includes an active blocking circuit 21 comprised of a resistor 22 in parallel with a series combination of a diode 25 and a transistor 26. Each of the control line wires 36 is operatively coupled to the transistor 26 of a respective active blocking circuit 21.

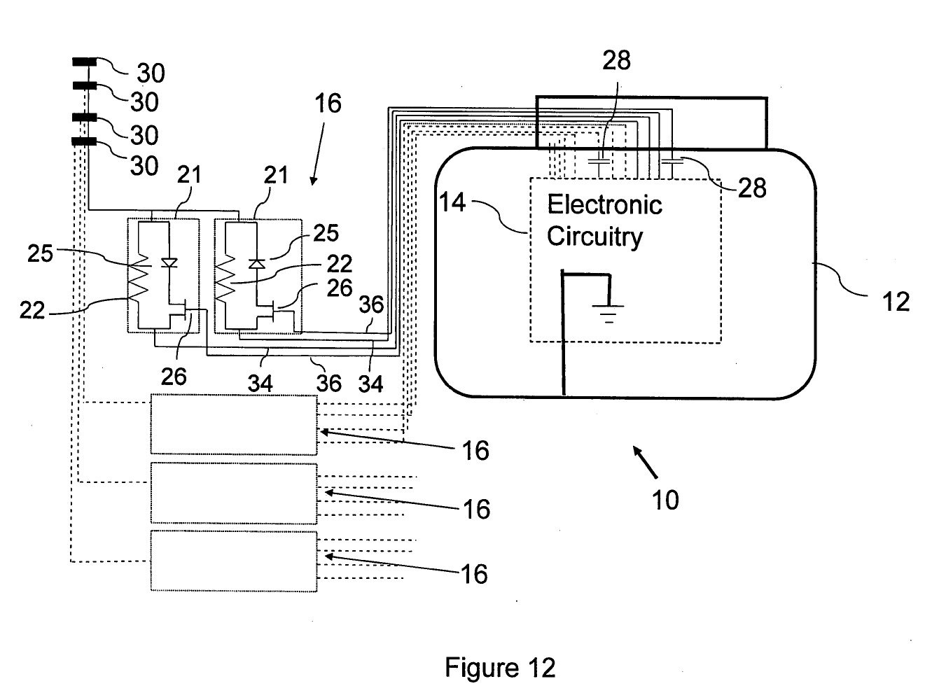

FIG. 12 is a schematic diagram of another particular multi-electrode two directional pacing TDC embodiment of the present invention, which shows an IPG 10 surrounded by a case 12, in which the IPG 10 is comprised of electronic circuitry 14 operatively coupled to four leads 16, in which each of the four leads 16 is operatively coupled to an electrode tip 30. Each of the four leads 16 is comprised of two signal wires 34 and two control line wires 36, in which each of the two signal wires 34 includes an active blocking circuit 21 comprised of a resistor 22 in parallel with a series combination of a diode 25 and a transistor 26. Each of the control line wires 36 is operatively coupled to the transistor 26 of a respective active blocking circuit 21.

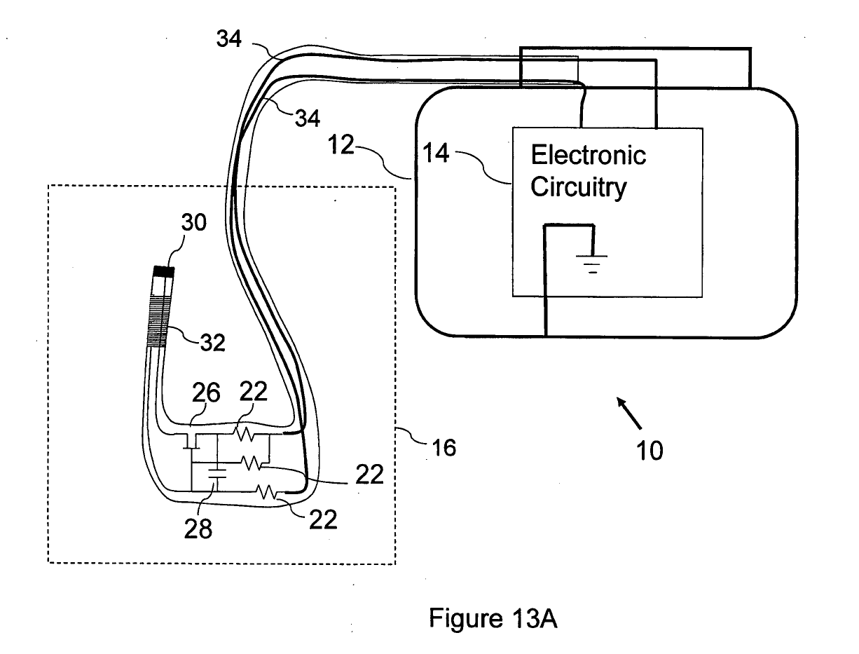

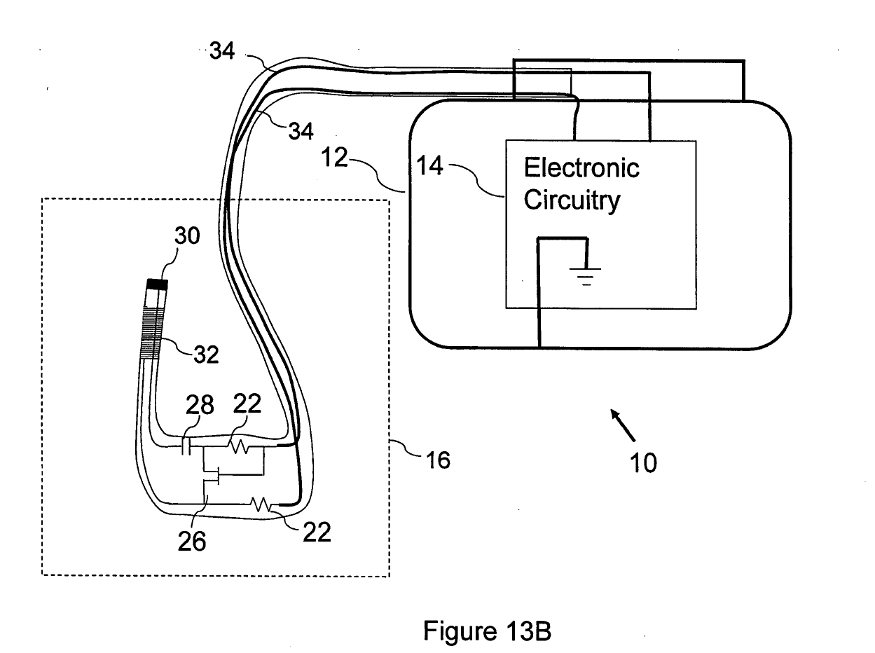

FIG. 13A is a schematic diagram of a Capacitor Switch Circuit (CSC)-Parallel embodiment of the present invention, which shows an implantable apparatus 10 (also referred to as an implantable pace generator) surrounded by a case 12, in which the implantable apparatus 10 is comprised of electronic circuitry 14 operatively coupled to one lead 16. The lead 16 includes two wires 34, in which one wire terminates in an electrode tip 30 and the other wire 34 terminates in a ring 32. The first wire 34 includes a resistor 22 in series with a transistor 26 and the second wire 34 includes a resistor 22. A capacitor 28 is operatively coupled between the first wire 34 and the second wire 34.

FIG. 13B is a schematic diagram of a Capacitor Switch Circuit (CSC)-Series embodiment of the present invention, which shows an implantable apparatus 10 surrounded by a case 12, in which the implantable apparatus 10 is comprised of electronic circuitry 14 operatively coupled to one lead 16. The lead 16 includes two wires 34, in which one wire terminates in an electrode tip 30 and the other wire 34 terminates in a ring 32. The first wire 34 includes a resistor 22 and a capacitor 28 in series and the second wire 34 includes a resistor 22. A transistor 26 is operatively coupled between the first wire 34 and the second wire 34. In addition, there is a control line wire 36 between the electronic circuitry 14 and the transistor 26.

In the embodiments shown in FIGS. 13A and 13B, when charging the capacitor 28, the connection between a body part of a patient and the capacitor 28 is kept open. The capacitor 28 is charged with the small current flowing on the resistors 22 between two pace pulses. In order to pace, the accumulated energy on the capacitor 28 discharges onto the body part. The duration is programmed to the implantable pace generator by the physician to achieve a desired treatment. Typically, the charging period is approximately one second, whereas the discharge period is about 1 msec. These embodiments are suitable only for bipolar pacing. As shown, the capacitor 28 can be placed in parallel or series to leads 16 and a PMOS transistor 26 may be selected as a switch in this embodiment.

In the CSC-Parallel embodiment, there are no control line wires. By using a suitable transistor, the pacing process easily can be adjusted. This embodiment, however, may experience “electrode charging” because the average current applied to the electrode is not zero.

In the CSC-Series embodiment, there is a control line wire in the lead of these embodiments. By applying a suitable control voltage on the transistor, the pacing process can be easily adjusted. Because there is a series capacitor in the circuit, “electrode charging” will not occur.

In both of the CSC embodiments, the capacitor and serial resistor values need to be optimized in order to have the most power efficient condition. Typically, PMOS (MOSFET) transistors have a very small resistance (in the order of 50Ω) when a transistor is on. Therefore, the transistor effect in the efficiency calculation can be ignored. During charging, some power will dissipate in the serial resistors and some power will be transmitted onto the body part of a patient. In order to increase power transmission to a body part, an efficiency equation from a system solution has been found.

After a system is activated, a capacitor voltage reaches its equilibrium point a few seconds later because of the capacitor charging and discharging duration period. At this point, the capacitor is charged by the battery. Its voltage value increases during a single charging period and reaches a specific value defined as V_{C2(0)}. Then the capacitor discharges on the body resistance. Its voltage value decreases during a single discharging period and reaches a specific value defined as V_{C1(0)}. A capacitor charging equation is obtained as follows:

$${V}_{C 2} ( 0 ) = {V}_{s} + ( {V}_{C 1} ( 0 ) - {V}_{s} ) {e}^{\frac{- {t}_{1}}{{R}_{1} C}} \qquad ( 1 )$$

where C is capacitance of capacitor of the model, t₁ is the capacitor charging duration (on the order of 1 sec), and R₁ is the total series resistance on the capacitor during charging period.

Similarly, a capacitor discharging equation is as follows:

$${V}_{C 1} ( 0 ) = {V}_{C 2} ( 0 ) {e}^{\frac{- {t}_{2}}{{R}_{2} C}} \qquad ( 2 )$$

where, C is capacitance of capacitor of model, t₂ is the capacitor discharging duration (on the order of 1 msec), and R₂ is the target body part resistance during the discharging period.

The two above-described equations are used to obtain efficiency equation. During charging, some power is dissipated on R₁. An equation of dissipated energy on serial resistance in one cycle is as follows:

$${W}_{1} = {\frac{C}{2} [ {V}_{s} - {V}_{C 2} ( 0 ) {e}^{\frac{- {t}_{2}}{{R}_{2} C}} ]}^{2} ( 1 - {e}^{\frac{- {2}_{{t}_{1}}}{{R}_{1} C}} ) \qquad ( 3 )$$

where W₁ is dissipated energy on serial resistance in one cycle.

During discharging, some power is transmitted on the body part of a patient and an equation of transmitted energy to body part in one cycle is as follows:

$${W}_{2} = \frac{C}{2} {V}_{C 2} ( 0 ) 2 ( 1 - {e}^{\frac{- {2}_{{t}_{2}}}{{R}_{2} C}} ) \qquad ( 4 )$$

where W₂ is transmitted energy to a body part in one cycle.

By using these two equations, an efficiency equation is obtained as follows:

$$= \frac{{W}_{2}}{{W}_{1}} = \frac{{( - 1 + {e}^{\frac{{t}_{1}}{{R}_{1} C}} )}^{2} ( 1 - {e}^{\frac{{2}_{{t}_{2}}}{{R}_{2} C}} )}{{( ( 1 - {e}^{\frac{- {t}_{2}}{{R}_{2} C}} ) {e}^{\frac{{t}_{1}}{{R}_{1} C}} )}^{2} ( 1 - {e}^{\frac{- {2}_{{t}_{1}}}{{R}_{1} C}} )} \qquad ( 5 )$$

where η is efficiency of the capacitor charging and discharging process.

This efficiency equation is solved by using MATLAB (The MathWorks Inc., Natick, Mass., USA) with respect to R₁ and C values.

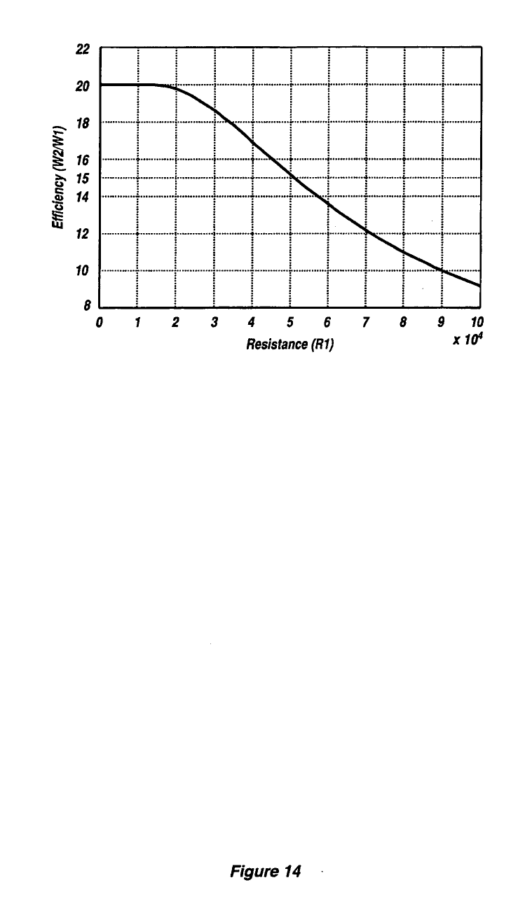

FIG. 14 is an efficiency plot of SPEC as a function of R₁, and shows efficiency plotted as a function of R₁ for C=10 μF, t₁=1 sec, t₂=1 msec, R₂=1KΩ.

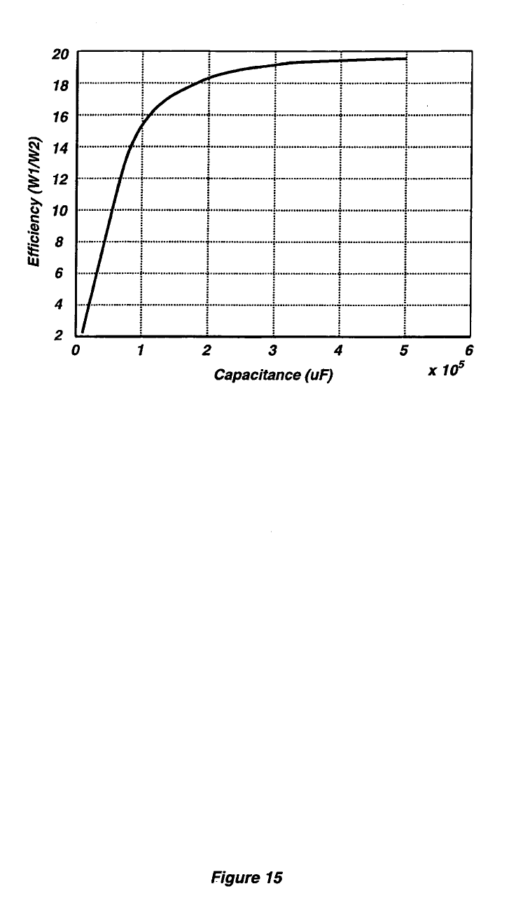

FIG. 15 is an efficiency plot of SPEC as a function of capacitance, and shows efficiency plotted as a function of capacitance for R₁=50 KΩ, t₁=1 sec, t₂=1 msec, R₂=1 KΩ.

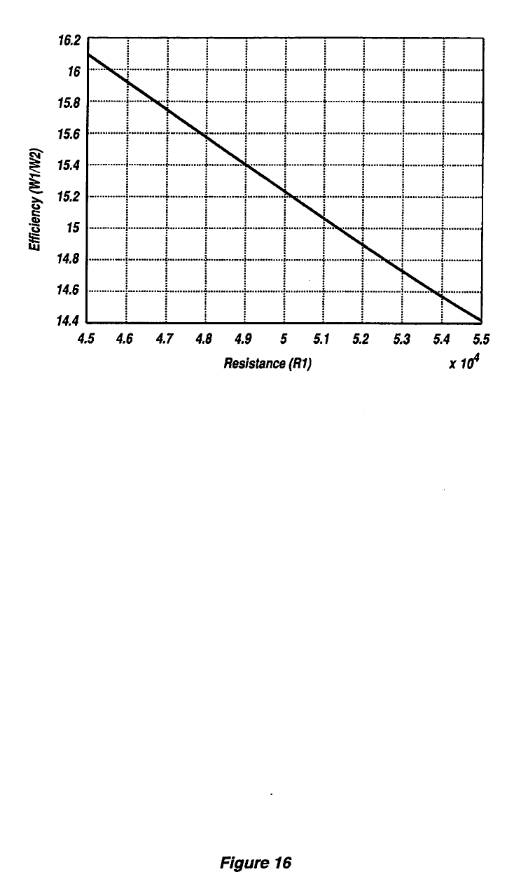

FIG. 16 is an efficiency plot of SPEC as a function of R₁, and shows efficiency plotted as a function of R₁ for C=10 μF, t₁=1 sec, t₂=1 msec, R₂=1 KΩ with the interval of 45 KΩ-55 KΩ.

According to this solution, capacitance was selected as C=10 μF and resistance selected as 50 KΩ. The efficiency was greater than 15 (η>=15) for these values. In order to block passage of RF current from the gate of MOSFET to tip and ring, 100 KΩ is put on the control line of a MOSFET transistor gate for both models. This resistance does not affect the pulse process because there is no current on the MOSFET transistor gate. Thus, DC voltage level on the control line is not changed with this resistance.

Additionally, charging duration and discharging duration may be programmed to an implantable pace generator by the physician to achieve a desired treatment.

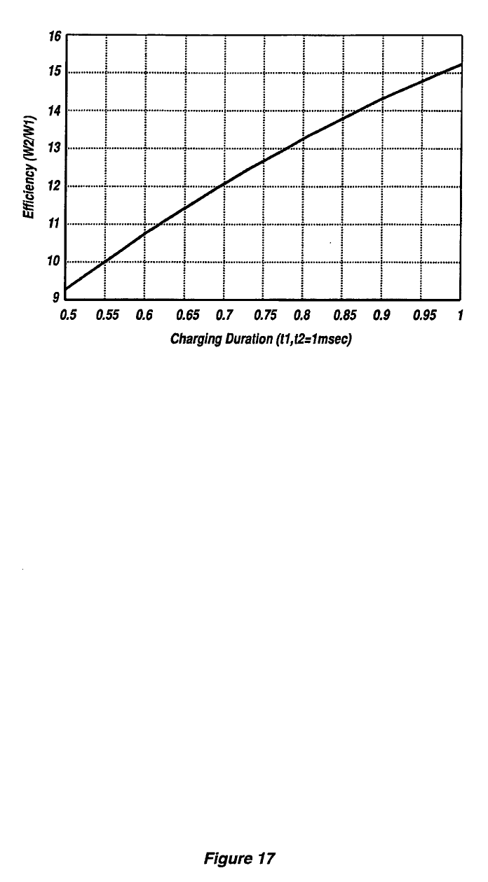

FIG. 17 is an efficiency plot of SPEC as a function of t₁, which shows efficiency plotted as a function of t₁ for C=10 μF, R₁=50 KΩ, t₂=1 msec, R₂=1 KΩ.

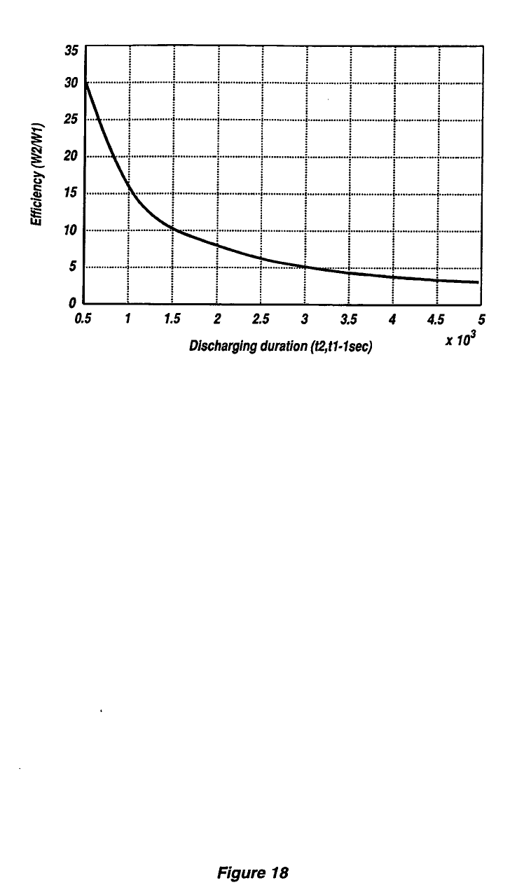

FIG. 18 is an efficiency plot of SPEC as a function of t₂, which shows efficiency plotted as a function of t₂ for C=10 μF, R₁=50 KΩ, t₂=1 msec, R₂=1 KΩ.

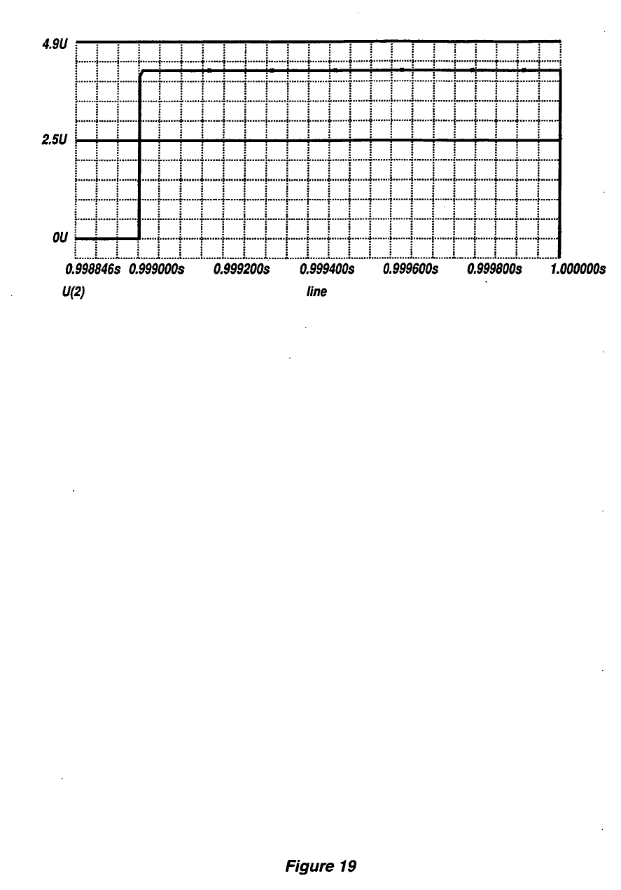

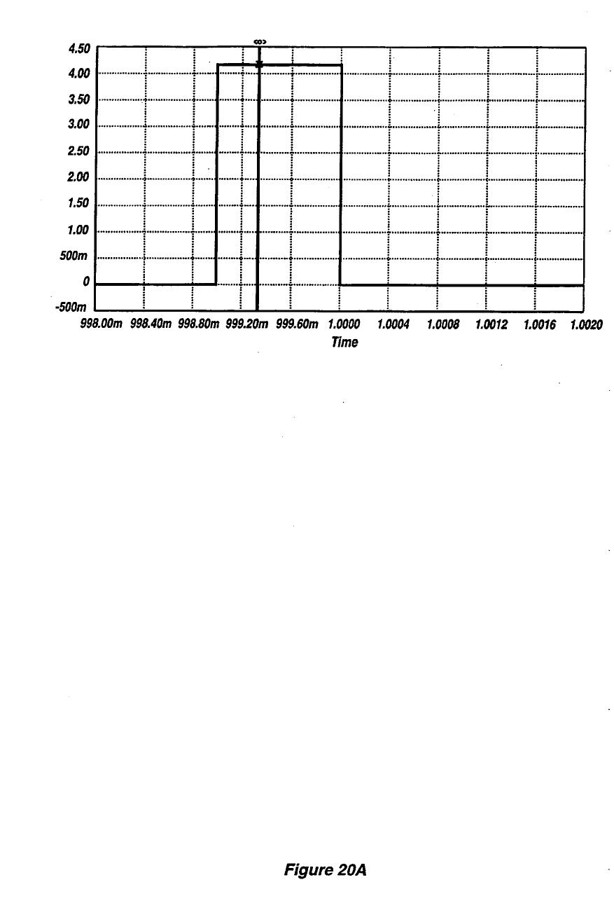

The plots shown in FIGS. 18 and 19 show the effect of charging and discharging duration on the efficiency of the system.

According to above calculations, specific resistance and capacitance values can be determined. In this design, modification is available when resistance distributes on a wire. Resistance of a wire can be calculated as follows:

$$R = \frac{ρ L}{A} \qquad ( 6 )$$

where ρ is resistivity, L is length, and A is the cross sectional area of the wire.

Based on the above calculation, optimum resistance is calculated as 25 KΩ on the signal end of the pace lead. The ground wire has the same resistance. It is assumed that the length of the wire is 50 cm and copper is used as a material. Resistivity of copper is ρ=1.724×10⁻⁸ ohm-m at 20 C.

By using above the data, the diameter of the copper wire is calculated as d=6.63×10⁻⁴ mm

In addition, another element can be used. If carbon is selected as a material (ρ=3.5×10⁻⁵ ohm-m), a new diameter value is calculated as d=2.986×10⁻² mm. These two different calculations show that a very thin wire can be produced and different materials, which are highly resistive, can be used in this design. Considering that there may be possible difficulties in constructing such thin wires with the desired mechanical properties, lumped circuit elements may be used to obtain the same electrical effect.

The present invention is more particularly described in the following non-limiting examples, which are intended to be illustrative only, as numerous modifications and variations therein will be apparent to those skilled in the art.

### EXAMPLES

### Example 1

### Simulations

### I. Pacing Pulse Models

Basic properties of simulations need to be defined before simulation results are provided. In standard stimulator applications, pulse duration changes with an interval of 0.1 ms to 2 ms. In this implementation, pulse duration is applied as 1 msec with a period of 1 sec. Target body part resistance is accepted as 1 KQ. Pulse level is applied as 5 volts.

a. Diode Resistor Circuit (DRC)

In order to simulate this implementation, ORCAD PSpice 9.1 Demo (Cadence, 2655 Seely Avenue, San Jose, Calif. 95134, USA) was used. In this simulation, Infineon Technologies BA595 pin diode model was used. A 1 msec pulse was applied in a period of 1 sec on the diode and resistor. As a result of this simulation, a 4.2 volt pulse level was observed on the tip (target body resistance) at 1 msec.

This result is shown in FIG. 19, which is a simulation result of a DRC embodiment on target body resistance. This result is a unipolar pacing result and one diode is used. For unipolar pacing, voltage level is easily calculated for a two diode threshold voltage. As a result of this simulation, a 3.6 volt pulse level is seen on the target body resistance at 1 msec.

b. Transistor Diode Circuit (TDC)

In order to simulate this implementation, a 5Spice Electronic Circuit Simulation Program (Non-commercial Version, Richard P. Andresen, 2004) was used. In this simulation, 1N5711 Schottky Barrier Diode was used as a diode model. PMOS and NMOS transistors were selected as a transistor model. In order to use the 5Spice Electronic Circuit Simulation Program, the properties of the PMOS and NMOS transistors need to be defined. These properties are given in Table 1.

TABLE 1
5Spice PMOS and NMOS models
NMOS 5SPICE MODEL
LEVEL = 3

| | | | | | |
|---|---|---|---|---|---|
| +TOX = | 7.9E−9 | NSUB = | 1E17 | GAMMA = | 0.5827871 |
| +PHI = | 0.7 | VTO = | 0.5445549 | DELTA = | 0 |
| +UO = | 436.256147 | ETA = | 0 | THETA = | 0.1749684 |
| +KP = | 2.055786E−4 | VMAX = | 8.309444E4 | KAPPA = | 0.2574081 |
| +RSH = | 0.0559398 | NFS = | 1E12 | TPG = | 1 |
| +XJ = | 3E−7 | LD = | 3.162278E−11 | WD = | 7.046724E−8 |
| +CGDO = | 2.82E−10 | CGSO = | 2.82E−10 | CGBO = | 1E−10 |
| +CJ = | 1E−3 | PB = | 0.9758533 | MJ = | 0.3448504 |
| +CJSW = | 3.777852E−10 | MJSW = | 0.3508721 | | |

PMOS 5SPICE MODEL
LEVEL = 3

| | | | | | |
|---|---|---|---|---|---|
| +TOX = | 7.9E−9 | NSUB = | 1E17 | GAMMA = | 0.4083894 |
| +PHI = | 0.7 | VTO = | −0.7140674 | DELTA = | 0 |
| +UO = | 212.2319801 | ETA = | 9.999762E−4 | THETA = | 0.2020774 |
| +KP = | 6.733755E−5 | VMAX = | 1.181551E5 | KAPPA = | 1.5 |
| +RSH = | 30.0712458 | NFS = | 1E12 | TPG = | −1 |
| +XJ = | 2E−7 | LD = | 5.000001E−13 | WD = | 1.249872E−7 |
| +CGDO = | 3.09E−10 | CGSO = | 3.09E−10 | CGBO = | 1E−10 |
| +CJ = | 1.419508E−3 | PB = | 0.8152753 | MJ = | 0.5 |
| +CJSW = | 4.813504E−10 | MJSW = | 0.5 | | |

In these simulations, a PMOS transistor is used in order to deliver current into the heart and an NMOS transistor is used in order to receive current from the heart. Simulations are made for unipolar and bipolar pacing.

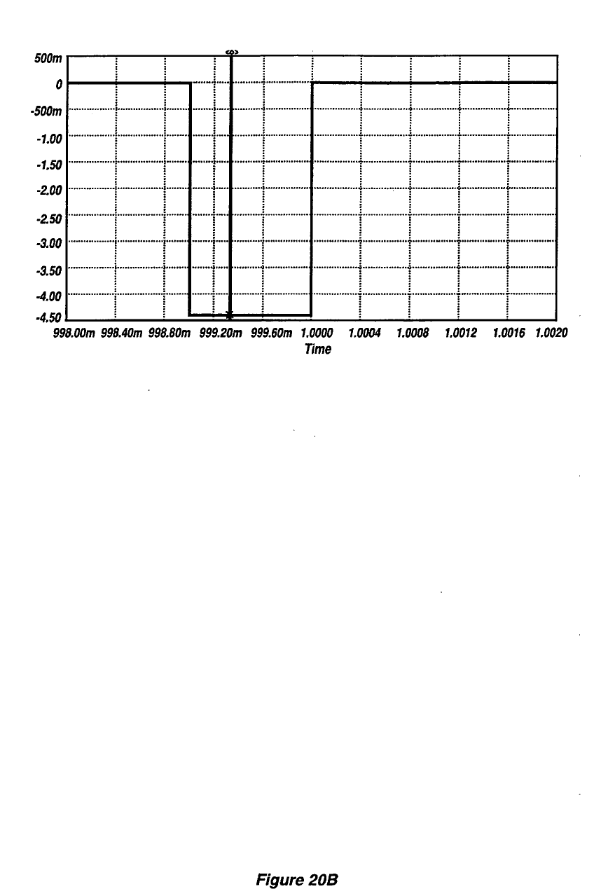

First, a unipolar pacing model was simulated. A series combination of a PMOS transistor and a diode were used in order to deliver current into the heart and a series combination of an NMOS transistor and a diode were used in order to receive current from the heart. A 1 msec pulse was applied in a period of 1 sec on the diode and resistor. As a result of this simulation, at a positive cycle, a 4.1 volt pulse level was observed and at a negative cycle, a −4.4 volt pulse was observed on the tip (target body resistance) at 1 msec. This result is shown in FIGS. 20A and 20B.

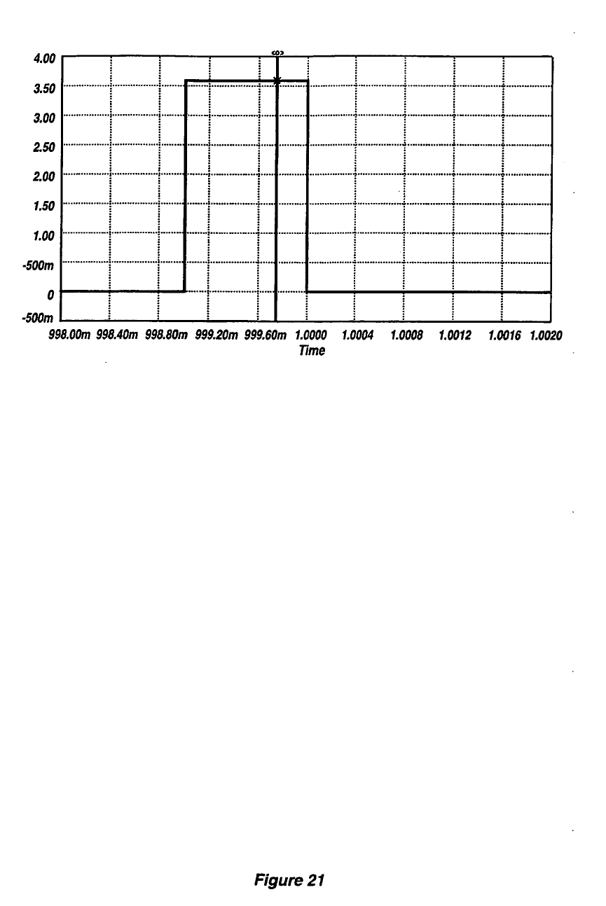

A bipolar pacing model was simulated by using a positive and negative current lines combination. A 1 msec pulse was applied in a period of 1 sec on the diode and resistor. As a result of this simulation, a 3.6 volt pulse level was observed on the target body resistance at 1 msec. This result is shown in FIG. 21.

c. Capacitor Switch Circuit (CSC)

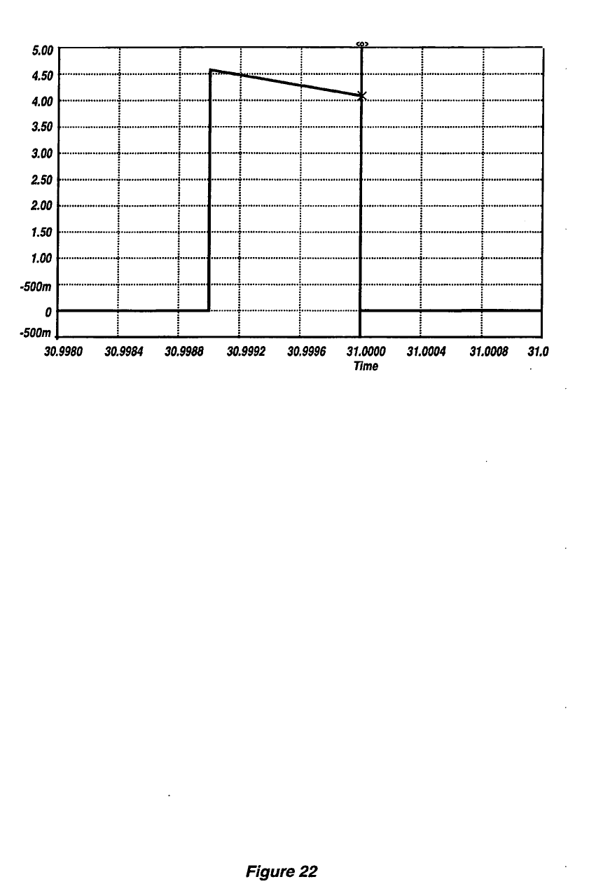

Capacitor Switch Circuit was simulated by using 5Spice Electronic Circuit Simulation Program (Non-commercial Version, Richard P. Andresen, 2004). Capacitor value was selected as 10 C=10 μF. Serial resistors were selected as 27 KΩ. By using these properties, CSC was simulated in a 5Spice Simulation Program. In this simulation, pulse level was selected as 5 volts. Pulse width of the source was selected as 999 ms for a charging period and a 1 msec source turn off. In 1 msec, a capacitor discharged on 1 KΩ target body resistance when a switching transistor was open. These results are shown in FIG. 22.

### II. Induced Voltage Signal Model

In these simulations, it was assumed that the pacemaker model of the present invention which contains an IPG and wires is placed in an MRI scanner. A 5 cm long IPG with a 35 cm length of wire are placed parallel in electric field in an MRI scanner.

a. Capacitor Switch Circuit (CSC) Simulation

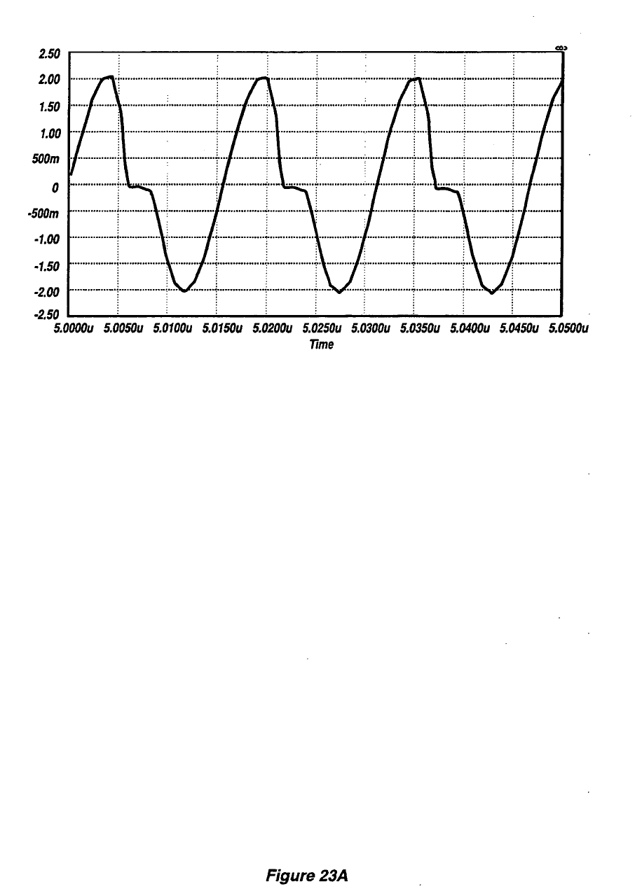

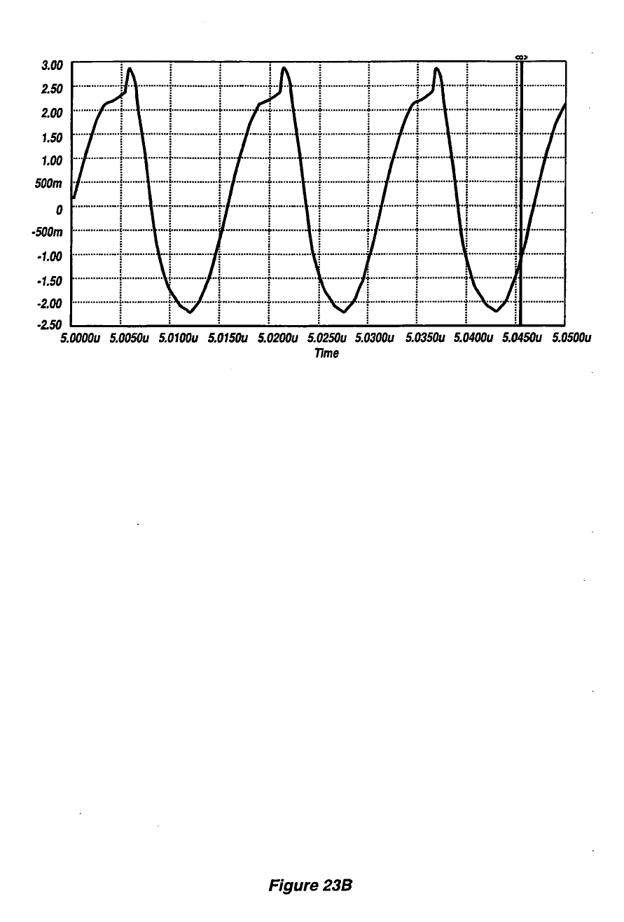

In this simulation, 27 KΩ resistors were placed on the leads. A 5Spice Electronic Circuit Simulation Program (Non-commercial Version, Richard P. Andresen, 2004) was used. A 53 volt peak induced voltage was applied as the worst case on the lead and a CSC-parallel capacitance model was added to it. As a result of this simulation, approximately a 2 volt peak induced voltage was observed on the tip (target body resistance) and approximately a 2.5 volt peak induced voltage was observed on the ring. These results are shown in FIGS. 23A and 23B.

Maximum peak voltage was approximately measured as 2 Vₚₑₐₖ on the tip. Approximate resistance was calculated as 25.5 KΩ, which is seen from a target body part. Maximum peak voltage was approximately measured as 2.5 Vₚₑₐₖ on the ring and approximate resistance was calculated as 20 KΩ, which is seen from a target body part. This is a significantly high resistance in order to block RF induction on the stimulator.

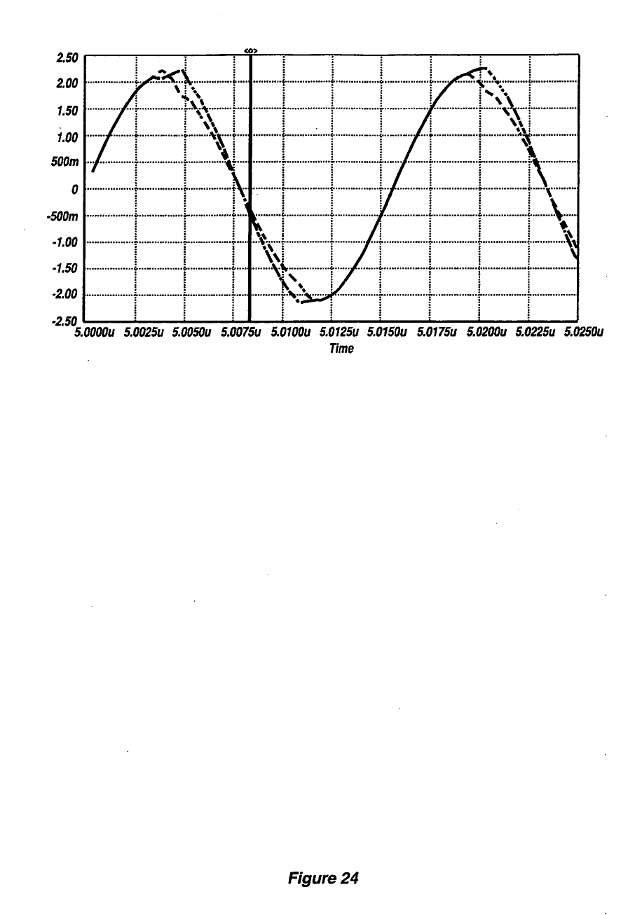

Along with a 53 volt peak induced voltage applied on the lead, a CSC-series capacitance model was added on to the lead. As a result of this simulation, approximately a 2.2 volt peak induced voltage was observed on the tip (target body resistance) and approximately a 2.2 volt peak induced voltage was observed on the ring. This result is shown in FIG. 24.

Maximum peak voltage was approximately measured as 2.2 Vₚₑₐₖ on the tip and ring. Approximate resistance was calculated as 23 KΩ, which is seen from a target body part. This is a significantly high resistance in order to block RF induction on the stimulator.

b. Transistor Diode Circuit (TDC) Simulation

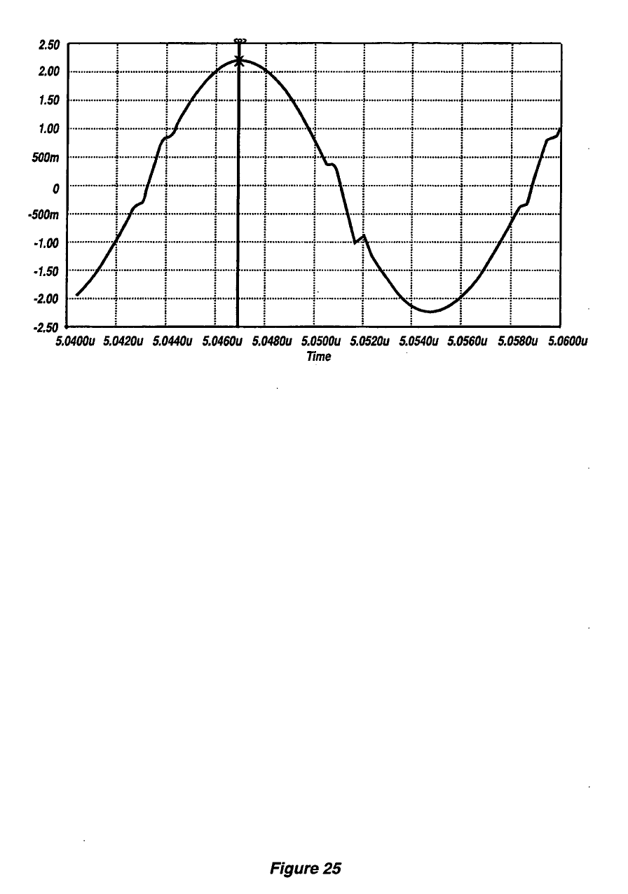

Along with a 53 volt peak induced voltage applied on the lead, TDC was added on to it. In this simulation, active circuits, which are connected to tip and ring, are symmetric. Therefore, simulation on two leads which connects to a tip gives the same result with leads which are connected to a ring. As a result of this simulation, approximately a 2.2 volt peak induced voltage was observed on the tip (target body resistance). This result is shown in FIG. 25.

Maximum peak voltage was approximately measured as 2.2 Vₚₑₐₖ on the tip and ring. Approximate resistance was calculated as 23.1 KΩm, which is seen from a target body part. This is significantly high resistance in order to block radio frequency induction on the stimulator.

### Example 2

### I. Experimental Methods

With the purpose of testing the CSC circuit, a circuit was designed that resembles an implantable pace generator (IPG). The simple pulse circuit contained a standard 9 Volt battery and a 16F84A 18-pin Enhanced FLASH/EEPROM 8-Bit microcontroller. The 16F84A microcontroller was programmed to generate a 1 msec pulse with a period of 1 sec. A 9 Volt battery and a 16f84A microcontroller were put on the same board and connected to each other. This board was put into a waterproof plastic box having dimensions of 9.5×5×2.5 (cm). Approximately 29 cm copper wires (0.5 mm diameter) were connected to this box for signaling and grounding. The waterproof plastic box was covered by copper tape and this conductive coating was connected to a ground wire.

In this circuit, a standard resistor value of 27 KΩ was used. A 500 KΩ resistor was connected to a gate of a PMOS transistor in order to have reliable gate control in the experiment. A 10 μF tantalum capacitor and a BS250 PMOS transistor were placed in the circuit. A CSC (parallel capacitor) implementation model was built by using these components.

The circuit was made waterproof. The circuit then was connected to an end of a 29 cm copper wire. The circuit length was approximately 2 cm and 4 cm copper wires were connected to an output side of this circuit. After all connections were completed, a total length of wire was approximately 35 cm. This design represents the safe implantable circuit of the present invention. In order to understand the safety performance of the CSC implementation model, an IPG was manufactured without an CSC circuit. The circuit was attached to 35 cm long wires to simulate electrodes.

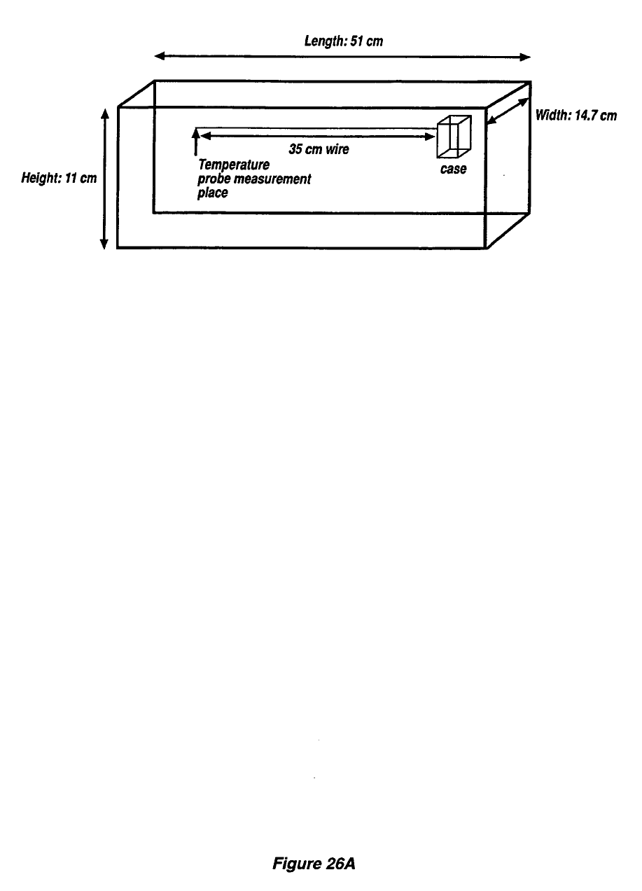

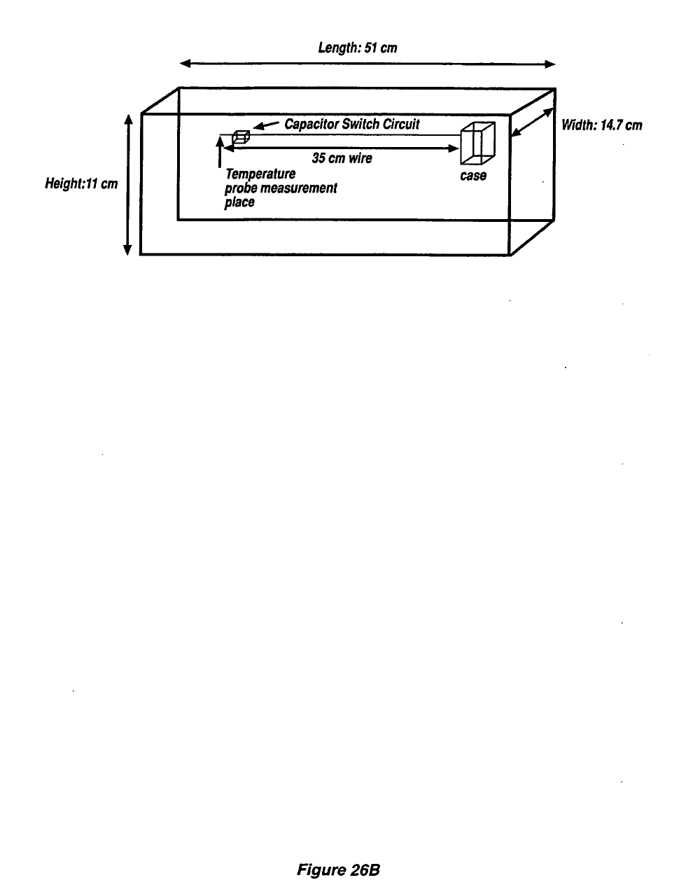

These two circuits were put into approximately rectangular plastic phantoms having dimensions of 51×14.7×11 (cm). These gel phantom setups are shown in FIGS. 26A and 26B.

In order to simulate tissue, peach jelly was selected (Dr. Oetker Gida Sanayi A. [symbol] , Fevzi [symbol] akmak Mah. Bekir Saydam Cad. No: 54 35865 Pancar—Torbali/İZMİR). Five liters water, 2 kg jelly and 50 gr. salt without iodine were put into a plastic case and then mixed together. Three hours later, a standard pulse circuit was put in one case and an energy stored pulse circuit was put in another case. After 1 day, the jelly solidified. By using this equipment, the following experiments were performed.

a. Heat Measurement Experiment

1. Capacitor Switch Circuit (CSC)

A heat measurement experiment was performed for CSC in GE Signa 1.5T MR scanner MRI Unit in Gazi Üniversitesi Tip Fakültesi (Gazi Hastanesi Be [symbol] evler/Ankara). In this experiment, a SPGR sequence was used with the following imaging parameters: Body coil, matrix 256×256, NEX: 4, TE: 4.0 msec, TR: 19 msec, flip angle: 17, bandwidth: 16.53 KHz, total scan time: 10.59 sec.

Scanner software estimated average SAR as 1.2328 W/Kg, and peak SAR as 2.4656 W/Kg. It should be noted that while the scanner software estimated these numbers, it assumes a person weighing 30 kg. However, the phantom used was quite different than the phantom and, therefore, the estimation method used in the scanner may not have displayed the correct number.

2. Diode Resistor Circuit (DRC)

Another experiment was conducted for a DRC. In this experiment, a Fast SPGR sequence was used with the following imaging parameters: Body coil (diameter: 60 cm), matrix 256×256, NEX: 4, TE: min, TR: 19 msec, flip angle: 90, bandwidth: 62.5 KHz, FOV: 48, Slice thickness: 20

Scanner software estimated average SAR as 1.26 W/Kg, and peak SAR as 2.51 W/Kg. It should be noted that while the scanner software estimated these numbers, it assumes a person weighing 30 kg. However, the phantom used was quite different than the first phantoms. It is a semi-cylinder with a radius of 10 cm and a length of 50 cm. The estimation method used in the scanner may not have displayed the correct number.

b. Nerve Stimulation Experiment

In order to test whether implanted devices increase the possibility of nerve stimulation in an MRI scanner during a routine MRI exam, a frog leg experiment was designed. A CSC design was tested with the same experimental set-up to show that this new design improves the safety of nerve stimulation safety of the implanted devices of the present invention.

The frog leg experiments were conducted in a GE Signa 1.5T MR scanner at Gazi University School of Medicine, Ankara, Turkey (Dr. Sevin Guney helped in the preparation of the frog experiment). In this experiment, the gastrocnemius nerve was used in which stimulation of the nerve can easily be seen with the naked eye. Preparation of the sciatic nerve for this experiment was as follows:

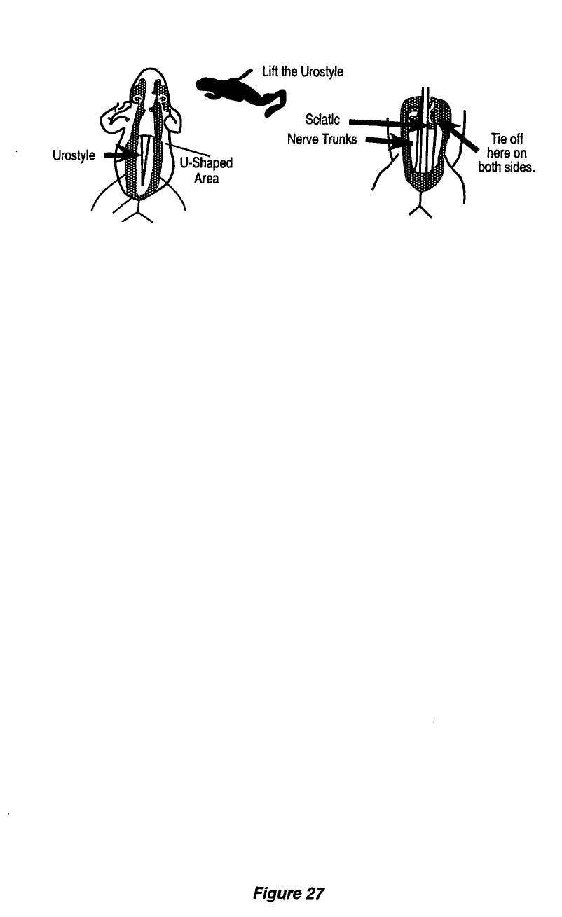

The frog was turned dorsal side up. The lower part of the thoracic cavity was cut (a U shaped area with the Urostyle down the center), taking care not to cut or extend the scissors too far into the cavity, as this would result in severing the sciatic nerve. The sciatic nerve was tied off with a length of thread just as it emerges from the spinal column. An illustration of this preparation is shown in FIG. 27.

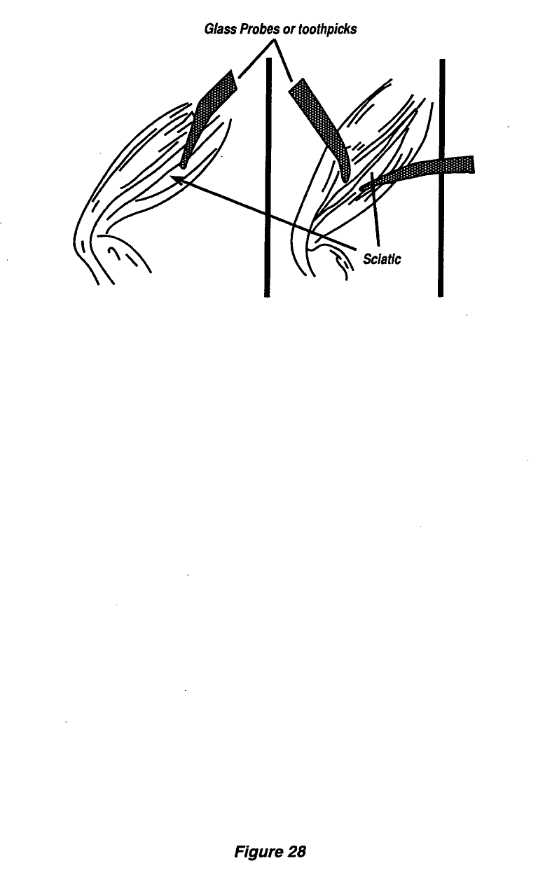

The sciatic nerve was exposed as it runs through the hip joint using blunt glass tools (i.e. two glass rods drawn out and fire-polished to a blunt 1 mm tip). The sciatic nerve runs down through the thigh musculature. This is shown in FIG. 28.

The sciatic nerve was freed from the spinal column down to the point that it enters the gastrocnemius. The nerve muscle preparation was placed in Ringer's solution. Ringer's solution is a solution of recently boiled distilled water which contains 8.6 gram sodium chloride, 0.3 gram potassium chloride, and 0.33 gram calcium chloride per liter. Ringer's solutions contains the chlorides of sodium, potassium, calcium and magnesium, in order to obtain a suitable physiological saline solution which can keep the leg of the frog alive outside of the body. Ringer's solution kept the prepared frog leg alive for more than two hours.

The next step was to find the threshold value of the nerve stimulation. For this purpose, a pulse generator was used. The pulse generator did not work properly because it's given values were not accurate. However, these measurements provided an overall understanding about the threshold value of the nerve stimulation. The frequency was adjusted to 1 Hz and the pulse width to 1 msec. The voltage value then was adjusted to 5 volts. On the frog's leg, stimulation was observed at this value. Voltage values then were decreased until stimulation was not observed on the frog's leg. From this measurement, it was concluded that the threshold level of the gastrocnemius nerve is approximately above 0.5 Volts.

The experimental setup was prepared by putting an IPG emulator into a plastic case.

This setup was filled with Ringer's solution and the frog's leg was put into this setup. The sciatic nerve of the frog's leg was connected to a lead of the emulator. Measurements were made with and without CSC. For both experiments all conditions were kept the same.

### II. Results

a. Heat Measurement Experiment

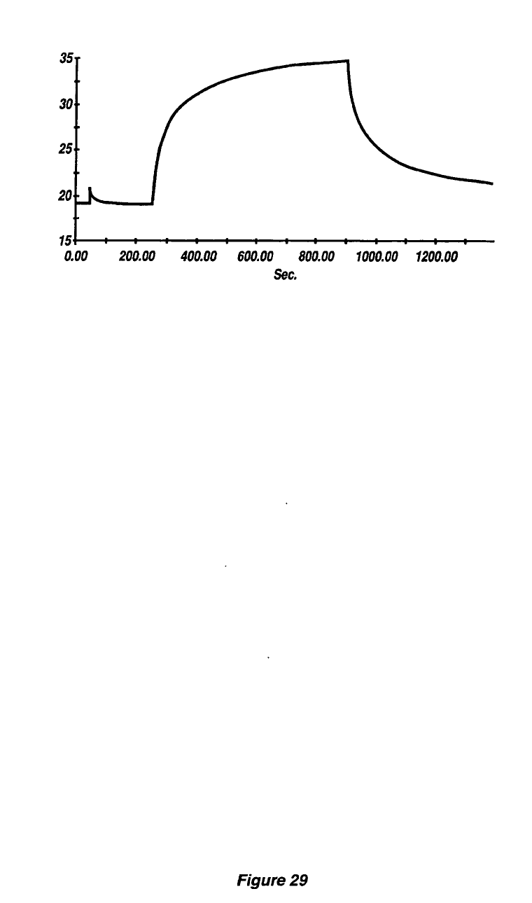

Two measurements were made for standard pulse circuit and a CSC in the same conditions as described above. The temperature was recorded using a FISO TMI Signal Conditioner and FOT-M fiberoptic temperature probes (FISO Technologies, Step. Foy, Quebec, Canada). The phantom was placed longitudinally in the MRI scanner. Its position was close to a body coil and this position was the same for both circuit measurements. A temperature probe was situated at a constant position at the end of the signal wire and placed longitudinally in the phantom. In the first measurement, a standard pulse circuit having a 35 cm copper wire was placed in the MRI scanner. The temperature increased more than 15° C. in 10 minutes. A measurement plot is shown in FIG. 29.

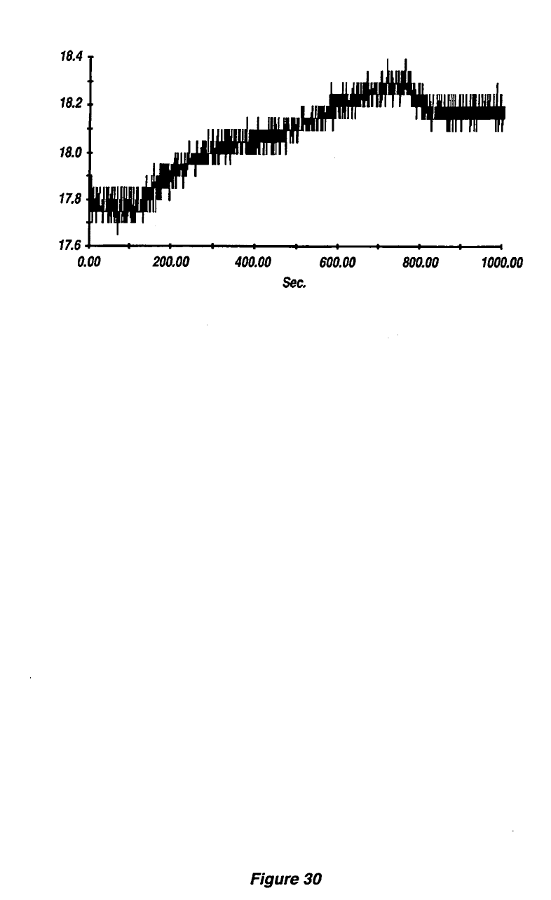

In the second measurement, a CSC was placed in an MRI scanner. In this measurement, the temperature increased approximately 0.5° C. in the same time period. This measurement plot is shown in FIG. 30.

In both plots, MRI sequence was started at 125 sec and finished at 750 sec after measurement initialized.

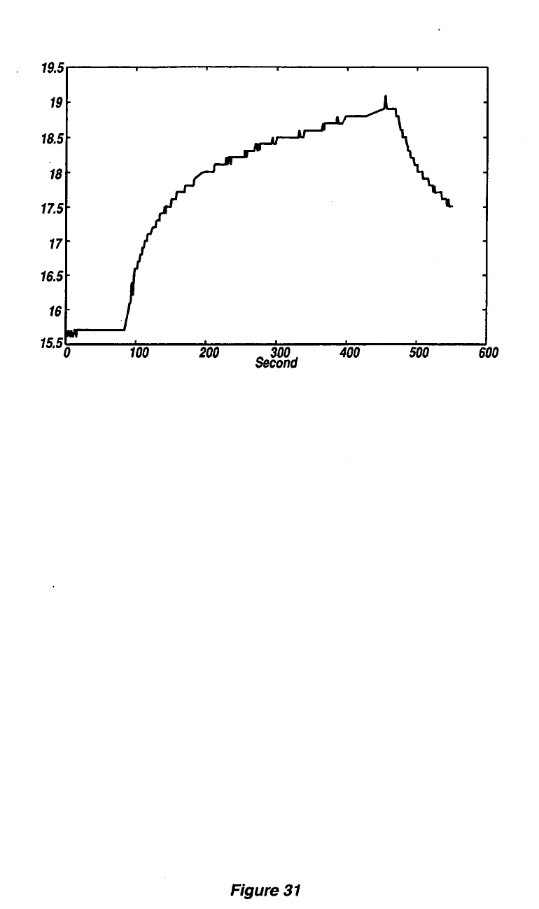

Measurements were made for a DRC in the conditions described above. The temperature was recorded using ReFlex-4 (Neoptix Inc., 1415, blvd Charest Quest, Suite 220, Quebec, QC, GIN 4N7, Canada) TMI Signal Conditioner and fiberoptic temperature sensor probes (Neoptix Inc., 1415, blvd Charest Quest, Suite 220, Quebec, QC, GIN 4N7, Canada). A phantom was placed longitudinally in the MRI scanner. Its position was close to a body coil. A temperature probe was situated at a constant position at the end of the tip and placed longitudinally in the phantom. In this experiment, an IPG model with a 30 cm copper wire was connected to the DRC and this was connected to 5 cm copper wire. This was placed in the MRI scanner. In this measurement, the temperature increase was approximately 3° C. in 6 minutes. This measurement plot is shown in FIG. 31.

b. Nerve Stimulation Experiment Results

In these experiments, a spin-echo single shot echo-planar sequence was used. For each of the designs, five different FOV values (5, 15, 31, 45 and 48 cm) were used. Changing FOV increases the amplitude of the readout gradients and, therefore, duration of the ramp time is increased (in GE scanners, the slope of the gradient waveform is kept constant). Therefore, low FOV values should result in longer exposure to the dB/dt. Other pulse sequence parameters were: matrix 256×256, TE: 95.4, TR: minimum, slice thickness: 7 mm, bandwidth: 20.63 KHz.

By using these properties, two measurements are made. For a simple pacemaker circuit with a 35 cm copper wire, stimulation of the frog's leg was observed for each FOV value. In contrast, in the second measurement with the CSC, no stimulation for each FOV value was observed. This experiment showed that the circuit was decreasing induced voltage lower than the threshold level of the gastrocnemius nerve.

### III. Discussion

Currently, the number of patients with active implants, such as deep brain stimulators (DBS) and cardiac stimulators, is very high and this number is expected to increase exponentially. On the other hand, MRI is an indispensable diagnostic tool for many diseases. Unfortunately, patients with active implants cannot be examined using MRI because the procedure carries a significant risk. Therefore, an MRI safe active implant design is of critical importance for a patient's health and for new treatment applications.

The inventors have proposed a new implantable apparatus and method to make an MRI safe implantable apparatus. This implantable apparatus uses active circuits on a lead in order to prevent induced currents on wires that comprise the lead. The inventors also have proposed case insulation in order to further reduce induced currents. Three main embodiments of this novel technique for an MRI safe implantable apparatus are presented herein. Each of them has different properties and can be preferred in different implementations. By simulations and heating experiments, it has been shown that the techniques used herein were successful in significantly reducing induced currents.

The manufacturing process of one main embodiment of the present invention, namely, a Diode Resistor Circuit (DRC), is rather straightforward as no tuning is necessary. Modern technology is able to produce very small sized diodes and resistors without much difficulty and without changing wire design.

The second main embodiment of the present invention, a Transistor Diode Circuit (TDC), is an advanced model of the DRC embodiment, and thus similar properties are provided in this embodiment. High impedance circuit elements are used at radio frequencies similar to DRC. In addition to this, TDC provides an additional contribution to the safety of stimulators because gradient induced current can be decreased using a TDC.

The third main embodiment, Capacitor Switch Circuit (CSC), is suitable for a bipolar pacing technique. Although miniaturization of this design may be more challenging than the other two embodiments, a CSC provides safety for both gradient and RF induced currents. The CSC embodiment can be implemented by using series and parallel capacitors. While a parallel capacitor design may suffer from a electrode charging problem, a series capacitor design uses additional control lines which avoids electrode charging.

In testing this new idea, only a CSC circuit was built. Because the CSC embodiment was shown effective in the above-described experiments, nonmagnetic elements were not used. CSC was constructed using a tantalum capacitor and a transistor which have magnetic properties. A very primitive pacemaker was constructed using a 16F84A 18-pin Enhanced FLASH/EEPROM 8-Bit microcontroller and 16V battery. These contained some magnetic materials, and thus these circuit elements may have added some error to the experiments.

Additionally, in the nerve stimulation experiment, the exact threshold level of the gastrocnemius nerve stimulation was not determined. Some parameters, such as gradient field amplitude and duration, were not measured during these experiments, and thus the nerve stimulation experiments were not enough to determine whether there was a significant nerve stimulation risk associated with implants. However, assuming there was a risk, the CSC embodiment was shown to be reduce this effect.

Although the three main embodiments of the present invention are suitable for most active implant designs, some active designs may require additional circuits. Implementation of these circuits are trivial given the above designs. For example, implantable cardioverter defibrillators (ICDs) require that circuit elements sustain large voltages and currents. Cardiac pacemakers will need additional circuits for sensing ECG signals.

Deep Brain Stimulation (DBS) is another basic area for stimulators. DBS delivers electrical stimulation signals to targeted areas in the brain. DBS is used, for example, for Parkinson's Disease, essential tremors and dystonia. DBS includes three parts: a lead, an IPG and an extension cable. DBS leads may have four or more electrodes. The extension cable is the connection point between the IPG and the DBS lead. The proposed active blocking circuits of the present invention may be placed outside the brain on the skull where the DBS lead is connected to the extension cable. The implementation of this design can be varied.

### IV. Conclusion

The present invention is related to the use of active blocking circuits in order to block induced currents on implant leads. Three main embodiments of the present invention have been described. In addition, there has been described a means of insulating the case of an IPG from the lead. The circuit designs of the present invention are novel over other possible designs found in the prior art. The use of active blocking circuits on one or more leads is demonstrated for the first time. These embodiments are very effective in blocking induced currents which may be due to radio frequencies and gradient fields. These embodiments may be implemented in a very small area without much difficulty and without requiring tuning.

While specific embodiments have been described in detail, it will be appreciated by those skilled in the art that various modifications and alternatives to those details could be developed in light of the overall teachings of the disclosure. Accordingly, the particular embodiments disclosed are meant to be illustrative only and not limiting as to the scope of the device and method described herein, which is to be given the full breadth of the appended claims and any and all equivalents thereof.

What is claimed is:

1. An apparatus that may be implanted within a patient's body which resists the induction of a current from an electromagnetic field external to said apparatus, comprising:
electronic circuitry for operating said apparatus; and
one or more leads operatively coupled to said electronic circuitry, said one or more leads each including one or more electrical wires and one or more active blocking circuits for resisting the induction of a current in said one or more leads from said electromagnetic field, each of said active blocking circuits being comprised of one or more electronic switches.

2. The apparatus of claim 1, wherein said electronic circuitry comprises an implantable pulse generator for generating one or more electrical pulses, and wherein each of said one or more leads delivers said one or more electrical pulses to tissue within the patient's body.

3. The apparatus of claim 1, wherein said one or more electronic switches each comprises a PIN diode.

4. The apparatus of claim 1, wherein said one or more electronic switches each comprises a transistor.

5. The apparatus of claim 3, wherein each of said one or more active blocking circuits is comprised of one of said one or more PIN diodes in parallel with a resistor.

6. The apparatus of claim 5, wherein said apparatus is comprised of one lead operatively coupled to an electrode, said one lead comprised of four wires, each of said four wires including one of said one or more active blocking circuits.

7. The apparatus of claim 5, wherein said apparatus is comprised of four leads, each of said four leads operatively coupled to an electrode, wherein each of said four leads is comprised of two wires, and wherein each of said two wires includes one of said one or more active blocking circuits.

8. The apparatus of claim 1, wherein each of said one or more active blocking circuits is comprised of a resistor in parallel with a series combination of a first one of said one or more electronic switches and a second one of said one or more electronic switches.

9. The apparatus of claim 8, wherein said first one of said one or more electronic switches is a diode and said second one of said one or more electronic switches is a transistor.

10. The apparatus of claim 9, wherein said apparatus is comprised of one lead operatively coupled to an electrode, said one lead comprised of four wires, wherein each of said four wires includes one of said one or more active blocking circuits, and wherein each of said four wires is operatively coupled to the transistor of the active blocking circuit of another one of said four wires.

11. The apparatus of claim 9, wherein said apparatus is comprised of four leads, each of said four leads operatively coupled to an electrode, wherein each of said four leads is comprised of two wires, wherein each of said two wires includes one of said one or more active blocking circuits, and wherein each of said two wires is operatively coupled to the transistor of the active blocking circuit of another one of said two wires from a different one of said four leads.

12. The apparatus of claim 9, wherein said apparatus is comprised of one lead, said one lead operatively coupled to an electrode, wherein said one lead is comprised of four signal wires and four control line wires, wherein each of said four signal wires includes one of said one or more active blocking circuits, and wherein each of said control line wires is operatively coupled to the transistor of a respective one of said one or more active blocking circuits.

13. The apparatus of claim 9, wherein said apparatus is comprised of four leads, each of said four leads operatively coupled to an electrode, wherein each of said four leads is comprised of two signal wires and two control line wires, wherein each of said two signal wires includes one of said one or more active blocking circuits and wherein each of said control line wires is operatively coupled to the transistor of a respective one of said one or more active blocking circuits.

14. The apparatus of claim 1, wherein at least one of said one or more leads is operatively coupled to an electrode, wherein at least one of said one or more leads includes a first wire, a second wire and a control line wire, said first wire including a resistor in series with a transistor, wherein said second wire includes a resistor, wherein a capacitor is operatively coupled between said first wire and said second wire, and wherein said control line wire is provided between said electronic circuitry and said transistor.

15. The apparatus of claim 1, wherein at least one of said one or more leads is operatively coupled to an electrode, wherein at least one of said one or more leads includes a first wire and a second wire, said first wire including a resistor and a capacitor in series, wherein said second wire includes a resistor, and wherein a transistor is operatively coupled between said first wire and said second wire.

16. An apparatus that may be implanted within a patient's body which resists the induction of a current from an electromagnetic field external to said apparatus, comprising:
electronic circuitry for operating said apparatus;
a case surrounding said electronic circuitry;
one or more leads operatively coupled to said electronic circuitry; and
an electronic switch provided between said electronic circuitry and said case for resisting the induction of a current in said one or more leads from said electromagnetic field.

17. The apparatus of claim 16, wherein said electronic circuitry comprises an implantable pulse generator for generating one or more electrical pulses, and wherein each of said one or more leads delivers said one or more electrical pulses to tissue within the patient's body.

18. A method of operating an implantable device which resists the induction of a current from an electromagnetic field external to the device, said device including electronic circuitry for operating the device, one or more leads operatively coupled to said electronic circuitry, each of said one or more leads being operatively coupled to a respective electrode, the method comprising:
periodically providing an electrical signal to a patient through each of said one or more leads and the electrode operatively coupled thereto; and
electrically isolating each of said electrodes from said electronic circuitry during a period in which the electrical signal is not being provided to said patient through each of said one or more leads.

19. The method of claim 18, wherein each of said electrodes are electrically isolated from said electronic circuitry by employing an active blocking circuit.
